(19) United States
(12) Patent Application Publication
(10) Pub. No.: US 2025/0205305 A1
(43) Pub. Date: Jun. 26, 2025

(54) SYSTEMS, METHODS, AND COMPOSITIONS FOR RESCUING PROTEIN MISFOLDING

(71) Applicant: The Trustees of Columbia University in the City of New York, New York, NY (US)

(72) Inventors: Alejandro Chavez, New York, NY (US); Samuel Resnick, New York, NY (US)

(21) Appl. No.: 18/833,802

(22) PCT Filed: Jan. 27, 2023
(86) PCT No.: PCT/US2023/061454

§ 371 Date: Jul. 26, 2024

Related U.S. Application Data
(60) Provisional application No. 63/303,777, filed on Jan. 27, 2022.

(51) Int. Cl. A61K 38/17; A61P 25/28

(52) U.S. Cl. CPC A61K 38/1709; A61P 25/28

(57) ABSTRACT

The present disclosure provides to systems, methods, and compositions for rescuing protein misfolding and preventing protein aggregation. Particularly the present disclosure provides methods and compositions comprising DNAJB6 or variants thereof, or polynucleotides encoding DNAJB6 or variants thereof. The present disclosure also provides methods for treating protein misfolding and/or protein aggregation diseases (e.g., multiple amyotrophic lateral sclerosis and frontotemporal dementia) by administering the systems or compositions to a subject in need thereof.

### CROSS-REFERENCE TO RELATED APPLICATIONS

This application claims the benefit of U.S. Provisional Application No. 63/303,777, filed Jan. 27, 2022, the content of which is herein incorporated by reference in its entirety.

### STATEMENT REGARDING FEDERALLY-SPONSORED RESEARCH OR DEVELOPMENT

This invention was made with government support under NS111851 awarded by the National Institutes of Health and AL190073 awarded by the Department of Defense. The government has certain rights in the invention.

### SEQUENCE LISTING STATEMENT

The contents of the electronic sequence listing titled 40177_601_SequenceListing.xml (Size: 12,159 bytes; and Date of Creation: Jan. 19, 2023) is herein incorporated by reference in its entirety.

### FIELD

The present invention relates to systems, methods, and compositions for rescuing protein misfolding and preventing protein aggregation, particularly methods and compositions comprising DNAJB6 or variants thereof, or polynucleotides encoding DNAJB6 or variants thereof for rescuing cellular toxicity and aggregation of RNA-binding proteins (e.g., FUS, TDP-43, and hnRNPA1).

### BACKGROUND

Human neurodegenerative diseases are a major source of morbidity and mortality worldwide and represent a significant unmet medical need. A hallmark of many neurodegenerative diseases (NDDs) is the intracellular accumulation of misfolded protein aggregates. To model the proteotoxicity imposed by NDD-associated proteins with an intrinsic propensity to aggregate such as Fused in Sarcoma (FUS), TAR DNA-binding protein (TDP-43), and alpha-synuclein, researchers have repeatedly turned to the yeast, Saccharomyces cerevisiae. In yeast, expression of these aggregation-prone proteins results in slow growth. By screening for genes that restore growth upon overexpression, researchers have been able to rapidly identify pathways involved in modulating the underlying proteotoxicity. By validating the hits from these yeast screens within mammalian systems, the results informed the understanding of disease pathobiology, and served as the basis for numerous therapeutic intervention strategies, some of which are being advanced by commercial entities.

While previous yeast-based studies have proven insightful, only a subset of NDD models have been screened using this approach. In addition, due to differences in the testing environment, genetic background, and method of screening, it is difficult to definitively compare results across studies in order to gain insight into common versus unique regulators of proteotoxicity. When comparisons between screens have been performed, few or no shared toxicity modifiers have been identified, even among related disease models such as TDP-43 and FUS. This lack of correlation conflicts with overlaps in clinical presentation between TDP-43 and FUS patients and the shared biochemical and biophysical properties of both proteins. Furthermore, as previous approaches are only able to study one model at a time, they have mainly focused on screening wild-type versions of NDD associated proteins, preventing our ability to address if patient mutations alter the underlying pathobiology and result in identification of differential proteotoxicity modifiers. Finally, most screens performed in yeast have searched for yeast genes that rescue the toxicity of NDD models, leading to identified hits with unclear or no known orthologous human counterpart to advance as a potential therapeutic candidate.

### SUMMARY

Provided herein are methods for rescuing RNA-binding protein misfolding or preventing RNA-binding protein aggregation comprising contacting the RNA-binding protein with DNAJB6 or an active fragment or variant thereof. In some embodiments, the RNA-binding protein comprises Fused in Sarcoma (FUS), TAR DNA-binding protein (TDP-43), heterogeneous nuclear ribonucleoprotein A1 (hnRNPA1), or a combination thereof.

In some embodiments, the RNA-binding protein is in a cell and the contacting comprises providing an effective amount of DNAJB6 or an active fragment or variant thereof, or a nucleic acid encoding the DNAJB6 or an active fragment or variant thereof to the cell. In some embodiments, the cell is a neuronal cell or glial cell.

In some embodiments, the cell is in a subject and the contacting comprises administering an effective amount of DNAJB6 or an active fragment or variant thereof, or a nucleic acid encoding the DNAJB6 or an active fragment or variant thereof to the subject.

In some embodiments, the methods further comprise decreasing the RNA-binding protein expression.

In some embodiments, the methods further comprise contacting the RNA-binding protein with an HSP70 family member protein or an active fragment or variant thereof.

Also provided herein are methods for treating a disease or disorder characterized by protein misfolding or protein aggregation comprising administering an effective amount of DNAJB6 or an active fragment or variant, or a nucleic acid encoding the DNAJB6 or an active fragment or variant to a subject in need thereof. In some embodiments, the disease or disorder is characterized by protein misfolding or protein aggregation of an RNA-binding protein. In some embodiments, the RNA-binding protein comprises FUS, TDP-43, hnRNPA1, or a combination thereof. In some embodiments, the methods further comprise decreasing the RNA-binding protein expression.

In some embodiments, the disease or disorder is a neurodegenerative disease. In some embodiments, the disease or disorder is selected from amyotrophic lateral sclerosis and frontotemporal dementia.

In some embodiments, the methods further comprise administering an HSP70 family member protein or an active fragment or variant thereof or a nucleic acid encoding the HSP70 family member protein or an active fragment or variant thereof.

In some embodiments, the DNAJB6 comprises an amino acid sequence having at least 70% (e.g., 75%, 80%, 85%, 90%, 95%, 98%, 99%) identity to SEQ ID NO: 1 or SEQ ID NO: 12. In some embodiments, the DNAJB6 comprises one or more mutations at positions 101, 107, 140, 160, 171, 182, and 192, as compared to SEQ ID NO: 1 or SEQ ID NO: 12. In some embodiments, the DNAJB6 comprises one or more mutations of R101, R107, F140, G160, G171, G182, and S192, as compared to SEQ ID NO: 1 or SEQ ID NO: 12. In some embodiments, the DNAJB6 comprises an amino acid sequence of SEQ ID NO: 1 or SEQ ID NO: 12.

Other aspects and embodiments of the disclosure will be apparent in light of the following detailed description.

### BRIEF DESCRIPTION OF THE DRAWINGS

FIG. 1 is a schematic outlining an exemplary method for simultaneously screening dozens of models at a time. Each colored yeast contains a unique DNA-barcode that is used to identify its abundance in the final pool using next generation sequencing. Each barcoded yeast is then associated with a particular aggregation-prone protein (e.g., FUS) or control protein (e.g., EYFP). All DNA-barcoded strains are then mixed together and in the non-induced state are able to maintain members at a consistent baseline level. Upon induction, in the presence of an inert control genetic modifier (e.g., mCherry), cells expressing an aggregation-prone protein grow poorly and this is seen as a sharp decrease in their barcode abundance. In contrast, cells expressing a non-toxic control gene such as EYFP continue to grow well. When the DNA-barcoded library is induced in the presence of an active genetic modifier that rescues one of the models in the pool, an improvement in growth of that model occurs and this is seen as an increase in its corresponding barcode abundance. The differences in the abundance of each model are compared between the control and genetic modifier conditions to identify novel interactions.

FIGS. 2A-2G show the screening of molecular chaperones from yeast and humans for their ability to rescue the proteotoxicity of various neurodegenerative disease and protein misfolding models. FIG. 2A is a chart of models included in screen and disease association. FIG. 2B shows log2 fold change plotted for interactions between selected yeast chaperones and models. Log2 fold changes are shown as an average of all barcoded strains associated with that model. FIG. 2C shows the statistically significant interactions between selected yeast chaperones and models. FIG. 2D is a validation of interactions between yeast chaperones and models. Data are shown as mean±s.d. for three biological replicates. FIG. 2E shows log2 fold change plotted for interactions between selected human chaperones and models FIG. 2F shows the statistically significant interactions between selected human chaperones and models. FIG. 2G is a validation of interactions between human chaperones and models. Data are shown as mean±s.d. for three biological replicates. Comparisons between two conditions were conducted with Welch's t tests while multiple comparisons were conducted with ordinary one-way ANOVA; ** P≤0.01, ***P≤0.001, ****P≤0.0001.

FIGS. 3A-3J show DNAJB6 is a rescuer of FUS, TDP-43, and hnRNPA1. FIG. 3A is a graph showing DNAJB6 rescues proteotoxicity of FUS, TDP-43, and hnRNPA1 in yeast. Data are shown as mean±s.d. for three biological replicates. FIGS. 3B-3D are western blots following overexpression of TDP-43, FUS, and hnRNPA1, respectively, in HEK293T cells resulting in formation of SDS-insoluble (RIPA buffer insoluble), urea-soluble species. The formation of these species can be reduced by co-expression of DNAJB6. FIG. 3E is a quantification of the urea soluble species normalized to total protein detected by Ponceau S staining in FIGS. 3B-3D. Each independent replicate was normalized to its associated EYFP rescued sample. FIG. 3F is validation of non-target control (NTC) and DNAJB6 KO lines. DNAJB6 has two isoforms; DNAJB6a and DNAJB6b, which are 36 kDa and 27 kDa in size, respectively. FIGS. 3G-3I are western blots following overexpression of TDP-43, FUS, and hnRNPA1 in DNAJB6 KO lines shows an increase in the propensity to form SDS-insoluble, urea-soluble species. FIG. 3J is the quantification of the urea soluble species normalized to total protein detected by Ponceau S staining in FIGS. 3G-3I. Each independent replicate was normalized its associated EYFP rescued sample. All data are shown as mean±s.d. for three biological replicates. All statistical tests were conducted with Welch's t tests; ns=not significant P>0.05, *P≤0.05, **P≤0.01, ***P≤0.001, ****P≤0.0001.

FIGS. 4A-4I show DNAJB6 modulates the dynamics of phase separated species. FIG. 4A shows that induction of stress granule formation using sodium arsenite (NaAsO₂) increases the formation of sarkosyl-insoluble TDP-43 in DNAJB6 KO over NTC control cells. FIG. 4B is the quantification of FIG. 4A. Data are shown as mean±s.d. for three biological replicates. A comparison between the proportion of TDP-43 in the urea fraction was determined with a Welch's t test; **P≤0.01. FIG. 4C shows DNAJB6 overexpression reduces the formation of sarkosyl-insoluble TDP-43 upon NaAsO₂ stress. FIG. 4D is the quantification of FIG. 4C. Data are shown as mean±s.d. for three biological replicates. A comparison between the proportion of TDP-43 in the urea fraction was determined with a Welch's t test; *P≤0.05. FIG. 4E is an image showing DNAJB6 at 3 μM concentration undergoes liquid-liquid phase separation (LLPS) at physiological salt concentrations. FIG. 4F are images showing AF555 labeled DNAJB6 intermixes with FUS-mEmerald condensates when mixed at equimolar concentrations (1.25 μM) at physiological salt concentrations. Scale bar represents 5 microns. FIG. 4G are images showing DNAJB6 modifies the size of FUS-mEmerald condensates upon co-incubation at equimolar concentrations (1.25 μM) at physiological salt concentrations. Condensates were imaged 30 min after mixing. Scale bar represents 10 microns. FIG. 4H is the quantification of FIG. 4G. Statistics were conducted with a Kruskall-Wallis test with Dunn's multiple comparison; ns=not significant P>0.05, ****P≤0.0001. FIG. 4I are images showing that the incubation of FUS-mEmerald (1 μM) for 48 hours at physiological salt concentrations results in the formation of large irregular aggregates. Incubation of FUS-mEmerald and DNAJB6 at equimolar concentrations (1 μM) at physiological salt concentrations prevents the formation of aggregates. Scale bar represents 5 microns.

FIGS. 5A-5E show that a Deep Mutational Scan (DMS) of DNAJB6 identifies potentiated variants. FIG. 5A is a schematic overview of DMS approach for testing DNAJB6 variants against the FUS model. A library of DNAJB6 mutants (as shown with SEQ ID NOs: 6-11) is generated using a site directed mutagenesis-based approach, the resulting plasmid library is transformed into yeast containing the FUS model and grown under inducing conditions, finally the relative growth rates of all mutants are determined and normalized to the wild type DNAJB6 variant. FIG. 5B is a DMS heatmap for residues 83-194 of DNAJB6. Intensity of blue or red colored boxes indicates increased or decreased activity as compared to the wild type DNAJB6 protein. * indicates stop codon mutation, black dots (⋅) mark the wild type residue for each site in the protein. FIG. 5C shows the validation of DMS results in the FUS model and testing of variants against the TDP-43 model. FIG. 5D shows the testing of potentiated DNAJB6 variants in mammalian cells for their ability to reduce SDS-insoluble, urea-soluble FUS species upon overexpression. FIG. 5E is the quantification of the urea soluble species normalized to total protein detected by Ponceau S staining in FIG. 5D. Each independent replicate was normalized against its associated EYFP rescued sample. All data are shown as mean s.d. for three biological replicates. Comparisons were conducted with ordinary one-way ANOVA with display of significant comparisons; *P≤0.05, **P≤0.01, ***P≤0.001, ****P≤0.0001.

FIGS. 6A-6D show the unbiased screening of human ORFs identifies additional rescuers of proteotoxicity for multiple models. FIG. 6A shows log2 fold changes for selected human ORFs from an unbiased screen. Log2 fold changes are shown as an average of all barcoded strains associated with that model. FIG. 6B is the validation of rescuer effects with yeast NDD models. FIG. 6C shows the testing of FUS rescuers from ORFeome in mammalian cells for their ability to reduce SDS-insoluble, urea-soluble FUS species upon overexpression. FIG. 6D is the quantification of the urea soluble species normalized to total protein detected by Ponceau S staining in FIG. 6C. Each independent replicate was normalized its associated EYFP rescued sample. All data are shown as mean±s.d. for three biological replicates. Comparisons between two conditions were conducted with Welch's t tests while multiple comparisons were conducted with ordinary one-way ANOVA; *P≤0.05, **P≤0.01, ***P≤0.001, ****P≤0.0001.

FIG. 7 shows a schematic of the development of a multiplexed screening platform. Left, Individual yeast strains containing an integrated DNA barcode are transformed with a proteotoxic expression construct before pooling. Middle, Rescuers are introduced en masse through mating and selection. Right, Mated barcode pools are grown in inducing media in 96-well plate format before DNA harvesting, NGS, and subsequent analysis.

FIGS. 8A-8D show en masse mating of a barcode pool and outgrowth of a mated barcoded pool do not perturb barcode ratios. FIG. 8A is an example of correlation plot between two separately mated pools that have been selected for diploids, each dot represents a different barcode within the population. FIG. 8B is correlation values for 36 comparisons between the barcode abundance for separately mated pools. FIG. 8C is an example of correlation plot between two separately mated pools that have been selected for diploids, and outgrown in inducing media, each dot represents a different barcode within the population. FIG. 8D is correlation values for 36 comparisons between separately mated and outgrown pools.

FIGS. 9A-9C show the comparison of three pooling strategies for detecting known interactions. FIG. 9A is log2 fold change heatmaps for 3 pooling strategies. Previously known interactions that are expected are outlined in purple. FIG. 9B is correlations between barcodes after pooled barcoded strains were mated to the same control rescuer, selected for diploids, and grown under inducing condition using each of the three different pooling strategies. FIG. 9C shows the coefficient of variation vs. relative barcode abundance plot for each of the 3 pooling strategies.

FIGS. 10A-10C show the exploration of biological and technical sources of error for the optimal screening strategy. FIG. 10A shows the correlation between biological replicates (separately mated, selected, outgrown, harvested, and PCR amplified) and technical replicates (same sample of harvested DNA separately PCR amplified). FIG. 10B shows the coefficient of variation vs. relative barcode abundance plot for biological replicates of pooled DNA-barcoded library mated to an inert rescuer demonstrating the effect of averaging between biological replicates. FIG. 10C shows the coefficient of variation vs. relative barcode abundance plot for technical replicates of pooled DNA-barcoded library mated to an inert rescuer demonstrating effect of averaging between technical replicates.

FIGS. 11A-11G show the validation of the optimized multiplexed screening approach using known genetic interactions. FIG. 11A shows that optimized conditions enable detection of positive control interactions in pilot screen. FIG. 11B-11G are spot assays validating tested interactions in pilot multiplexed screen, as indicated.

FIG. 12 shows a simulation of how redundant barcoding enhances the ability to reject the null hypothesis. Fold change required for rejection of null hypothesis simulated using optimized pooling conditions with two biological replicates and two technical replicates.

FIGS. 13A-13B show the determination of number of reads required to adequately sample 302-member DNA-barcoded pool. FIG. 13A shows the coefficient of variation vs. relative barcode abundance for DNA-barcoded library mated to an inert rescuer at different levels of read subsampling. FIG. 13B shows the number of individual barcoded strains with less than 10 raw reads at different levels of read subsampling.

FIGS. 14A-14B show a full yeast chaperone screen. FIG. 14A shows log2 fold change interactions between all tested yeast chaperones and the models included in the pool. FIG. 14B shows significant interactions between yeast chaperones and models included in the pool.

FIGS. 15A-15B show a full human chaperone screen. FIG. 15A shows log2 fold change interactions between all tested human chaperones and the models included in the pool. FIG. 15B shows significant interactions between human chaperones and models included in the pool.

FIGS. 16A-16F show validation of liquid culture growth assay. The interactions tested in the pilot screen for mCherry (FIG. 16A), alpha-synuclein (FIG. 16B), FUS (FIG. 16C), TDP-43 (FIG. 16D), RNQ1 (FIG. 16E), and SUP-35 (FIG. 16F) and validated with spot assays were re-tested with the liquid culture growth assay to validate its behavior. Data are shown as mean±s.d. for three biological replicates. Comparisons were conducted with ordinary one-way ANOVA with display of comparisons of interactions with positive changes in relative growth; ns=not significant, *P≤0.05, **P≤0.01, ***P≤0.001, ****P≤0.0001.

FIG. 17 shows that the subsampling of barcodes per model demonstrates power of redundant barcoding. Yeast and human rescuers with called hits were reanalyzed with fewer number of barcoded strains included in the analysis. All hits shown in the 5-7 Barcodes/Model condition were validated.

FIGS. 18A-18C show that DNAJB6 shows specific activity against FUS, TDP-43, and hnRNPA1. DNAJB6 was tested alongside other human HSP40 proteins for their ability to rescue FUS (FIG. 18A), TDP-43 (FIG. 18B), and hnRNPA1 (FIG. 18C) proteotoxicity in yeast. Comparisons were conducted with ordinary one-way ANOVA with display of comparisons of interactions with positive changes in relative growth; *P≤0.05, ***P≤0.001, ****P≤0.0001.

FIGS. 19A-19B show that RNA-seq demonstrates that DNAJB6 is significantly upregulated in response to FUS or TDP-43 overexpression in HEK293T cells. Volcano plots for HEK293T expressed chaperones for FUS (FIG. 19A) and TDP-43 (FIG. 19B) compared to EYFP overexpressing cells. Two biological replicates were done for each condition.

FIG. 20 shows that knockout of DNAJB6 does not impact SDS solubility of endogenously expressed FUS, TDP-43, or hnRNPA1 in HEK293T cells. HEK293T NTC and DNAJB6 KO cells were transfected with an EYFP expression vector and endogenous levels of TDP-43, FUS, and hnRNPA1 were assessed.

FIG. 21 shows that the effect of sodium arsenite on endogenous FUS, TDP-43, and hnRNPA1 solubility in HEK293T cells. HEK293T cells were exposed to stress for 2 h and were harvested with a tripartite extraction method to probe the solubility of FUS, TDP-43, and hnRNPA1.

FIGS. 22A-22B show that DNAJB6 accelerates a transition from liquid to gel-like condensates early after co-mixing. FIG. 22A is images of freshly formed FUS condensates co-incubated at equimolar concentrations (1.25 μM) at physiological salt concentrations with bovine serum albumin (BSA) or DNAJB6 and subjected to FRAP 30 minutes after incubation. Scale bar represents 5 microns, all images are at the same magnification FIG. 22B is the quantification of FIG. 22A.

FIGS. 23A-23B show identification of DNAJB6 domains important for activity in yeast. FIG. 23A shows the results of testing domain deletions and J domain H31Q loss of function point mutant for their ability to rescue the FUS expressing yeast model. ΔJ, ΔG/F, ΔS represent deletion of the J-domain, glycine-phenylalanine rich, or serine rich region of DNAJB6, respectively. FIGS. 23B confirms the expression of DNAJB6 and mutant variants. Comparisons were conducted with ordinary one-way ANOVA; ns=not significant, ***P≤0.001, ****P≤0.0001.

FIGS. 24A and 24B show the correlation statistics between biological replicates of deep mutational scan of DNAJB6. FIG. 24A is the correlation between log2 fold changes for all amino acid changes at all positions tested in the deep mutational scanning approach. FIGS. 24B is the correlation between log2 fold changes for amino acids in each set of 14 amino acids analyzed together in one sequencing batch.

FIG. 25 shows that increasing the amount of FUS plasmid transfected increases the amount of SDS-insoluble, urea-soluble species. Different doses of FUS expression plasmid were transfected and protein was harvested 48 h after transfection to assess the solubility of FUS.

FIGS. 26A-26C show the comparison of DNAJB6 isoform a (DNAJB6) with DNAJB6 isoform b (DNAJB6b). FIG. 26A is a graph showing DNAJB6 and DNAJB6b both rescue FUS proteotoxicity in yeast. Data are shown as mean±s.d. for three or six biological replicates. FIG. 26B shows that DNAJB6 and DNAJB6b isoforms and variants perform similarly in modulating the growth of the FUS proteotoxicity yeast model as determined and normalized to the wild type DNAJB6 and DNAJB6b, as indicated. Data are shown as mean±s.d. for three biological replicates. FIG. 26C are graphs quantifying the urea soluble species normalized to total protein in response to FUS, TDP-43, or hnRNPA1 overexpression in the presence of either control EYFP, DNAJB6 or DNAJB6b, as indicated, in mammalian cultures. Each independent replicate was normalized against its associated EYFP rescued sample. All data are shown as mean s.d. for three biological replicates. Comparisons were conducted with ordinary one-way ANOVA with display of significant comparisons; *P≤0.05, **P≤0.01, ***P≤0.001, ****P≤0.0001.

### DETAILED DESCRIPTION

The disclosed systems, compositions, and methods advance methods for rescuing protein misfolding and preventing protein aggregation.

The accumulation of misfolded proteins within intracellular aggregates is a distinctive feature observed within multiple neurodegenerative diseases (NDDs). However, the pathways that regulate protein misfolding, aggregation, and cellular toxicity remain poorly understood. Described herein is a platform that enabled simultaneous screening of dozens of NDD-associated proteins to rapidly uncover genetic modifiers that alter their solubility and toxicity. The human HSP40 chaperone, DNAJB6, was identified as a potent rescuer of the misfolding and proteotoxicity of multiple RNA-binding proteins implicated in ALS and FTD including FUS, TDP-43, and hnRNPA1. DNAJB6 was shown to have an intrinsic ability to phase separate under near-physiologic conditions and can alter FUS condensates by maintaining them in a gel-like state over long periods, preventing FUS aggregation. Domain mapping and deep mutational scanning on DNAJB6 provided identified a series of novel variants with enhanced activity.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The terms “non-naturally occurring,” “engineered,” and “synthetic” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

The term “amino acid” or “any amino acid” as used here refers to any and all amino acids, including naturally occurring amino acids (e.g., a-amino acids), unnatural amino acids, modified amino acids, and non-natural amino acids. It includes both D- and L-amino acids. Natural amino acids include those found in nature, such as, e.g., the 23 amino acids that combine into peptide chains to form the building-blocks of a vast array of proteins. These are primarily L stereoisomers, although a few D-amino acids occur in bacterial envelopes and some antibiotics. The “non-standard,” natural amino acids include, for example, pyrolysine (found in methanogenic organisms and other eukaryotes), selenocysteine (present in many non-eukaryotes as well as most eukaryotes), and N-formylmethionine (encoded by the start codon AUG in bacteria, mitochondria, and chloroplasts). “Unnatural” or “non-natural” amino acids are non-proteinogenic amino acids (e.g., those not naturally encoded or found in the genetic code) that either occur naturally or are chemically synthesized. Over 140 unnatural amino acids are known and thousands of more combinations are possible. Examples of “unnatural” amino acids include β-amino acids (β³ and β²), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, alpha-methyl amino acids and N-methyl amino acids. Unnatural or non-natural amino acids also include modified amino acids. “Modified” amino acids include amino acids (e.g., natural amino acids) that have been chemically modified to include a group, groups, or chemical moiety not naturally present on the amino acid. According to certain embodiments, a peptide inhibitor comprises an intramolecular bond between two amino acid residues present in the peptide inhibitor. It is understood that the amino acid residues that form the bond will be altered somewhat when bonded to each other as compared to when not bonded to each other. Reference to a particular amino acid is meant to encompass that amino acid in both its unbonded and bonded state. For example, the amino acid residue homoSerine (hSer) in its unbonded form may take the form of 2-aminobutyric acid (Abu) when participating in an intramolecular bond according to the present invention.

For the most part, the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14(2), (1975). To the extent that the names and abbreviations of amino acids and aminoacyl residues employed in this specification and appended claims differ from those suggestions, they will be made clear to the reader.

Throughout the present specification, unless naturally occurring amino acids are referred to by their full name (e.g., alanine, arginine, etc.), they are designated by their conventional three-letter or single-letter abbreviations (e.g., Ala or A for alanine, Arg or R for arginine, etc.). The term “L-amino acid,” as used herein, refers to the “L” isomeric form of a peptide, and conversely the term “D-amino acid” refers to the “D” isomeric form of a peptide (e.g., Dphe, (D)Phe, D-Phe, or ^{D}F for the D isomeric form of Phenylalanine). Amino acid residues in the D isomeric form can be substituted for any L-amino acid residue, as long as the desired function is.

In the case of less common or non-naturally occurring amino acids, unless they are referred to by their full name (e.g. sarcosine, ornithine, etc.), frequently employed three-or four-character codes are employed for residues thereof, including, Sar or Sarc (sarcosine, i.e. N-methylglycine), Aib (α-aminoisobutyric acid), Dab (2,4-diaminobutanoic acid), Dapa (2,3-diaminopropanoic acid), γ-Glu (γ-glutamic acid), Gaba (γ-aminobutanoic acid), β-Pro (pyrrolidine-3-carboxylic acid), and 8Ado (8-amino-3,6-dioxaoctanoic acid), Abu (2-amino butyric acid), βhPro (β-homoproline), βhPhe (β-homophenylalanine) and Bip (β,β diphenylalanine), and Ida (Iminodiacetic acid).

As used herein, the term “percent sequence identity” refers to the percentage of nucleotides or nucleotide analogs in a nucleic acid sequence, or amino acids in an amino acid sequence, that is identical with the corresponding nucleotides or amino acids in a reference sequence of the present disclosure after aligning the two sequences and introducing gaps, if necessary, to achieve the maximum percent identity. Hence, in case a nucleic acid or protein is longer than a reference sequence, additional nucleotides or amino acids that do not align with the reference sequence are not taken into account for determining sequence identity. A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510(2002)) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

A “vector” or “expression vector” is a replicon, such as plasmid, phage, virus, or cosmid, to which another nucleic acid segment, e.g., an “insert,” may be attached or incorporated so as to bring about the replication of the attached segment in a cell.

A cell has been “genetically modified,” “transformed,” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. For example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of proteins, nucleic acids, or compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods provided herein, the mammal is a human.

The term “contacting” as used herein refers to bring or put in contact, to be in or come into contact. The term “contact” as used herein refers to a state or condition of touching or of immediate or local proximity. Contacting a composition to a target destination, such as, but not limited to, an organ, tissue, or cell, may occur by any means of administration known to the skilled artisan.

As used herein, the terms “providing,” “administering,” and “introducing,” are used interchangeably herein and refer to the placement of the disclosed proteins, polypeptides, nucleic acids and polynucleotides into a cell, organism, or subject by a method or route which results in at least partial localization to a desired site. The administration can be by any appropriate route which results in delivery to a desired location in the cell, organism, or subject.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Protein Misfolding and Aggregation

Modeling of neurodegeneration in simplified cellular systems has yielded numerous fundamental insights as to how proteotoxic aggregation-prone species disrupt cellular function. Herein is disclosed a multiplexed approach in which numerous models can be screened in parallel, increasing the richness of the data obtained. By inserting the same model into several uniquely barcoded strains and analyzing their collective behavior, assay sensitivity and specificity was greatly enhanced. The quantitative nature of the approach better enabled capture of mild changes in growth by a putative rescuer as compared to traditional semi-quantitative methods. By simultaneously studying multiple models within the same genetic background and under the same testing paradigm, broad observations were able to be made about the nature of rescuers and the relationship between models.

As such, disclosed herein is a system or platform and method for screening modifiers of NDD-associated proteins. The systems and methods comprise a) a library of yeast comprising a plurality of populations of yeast each comprising a unique identifier (e.g., a DNA barcode) and a nucleic acid encoding an aggregation-prone protein or a control protein under control of an inducible promoter and b) one or more yeast strains comprising a nucleic acid encoding a putative modifier of the aggregation-prone protein.

In some embodiments, the methods comprise obtaining the library of yeast comprising a plurality of populations of yeast each comprising a unique identifier and a nucleic acid encoding an aggregation-prone protein or a control under control of an inducible promoter; mating the library of yeast with one or more yeast strains comprising a nucleic acid encoding a putative modifier of the aggregation-prone protein; culturing diploid yeast comprising the nucleic acid encoding an aggregation-prone protein and the nucleic acid encoding the putative genetic modifier under conditions which induce the expression of the aggregation-prone protein; measuring the quantity, concentration or level of the identifier to determine the abundance of each of the plurality of populations of yeast; and, optionally, identifying a modifier as those putative modifiers associated with an increase of barcode abundance.

In some embodiments, the methods further comprise generating the library of yeast comprising a plurality of populations of yeast each comprising a unique identifier and a nucleic acid encoding an aggregation-prone protein or a control under control of an inducible promoter, generating the yeast strains comprising a nucleic acid encoding a putative modifier of the aggregation-prone protein, and/or selecting for diploid yeast strains comprising the nucleic acid encoding an aggregation-prone protein and the putative modifier. Measuring the quantity, concentration or level may comprise one or more of DNA isolation, PCR amplification, and DNA sequencing.

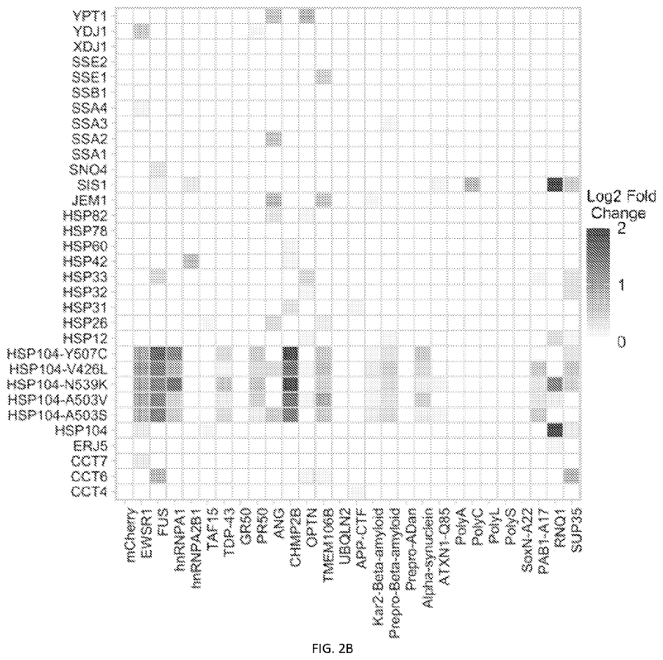

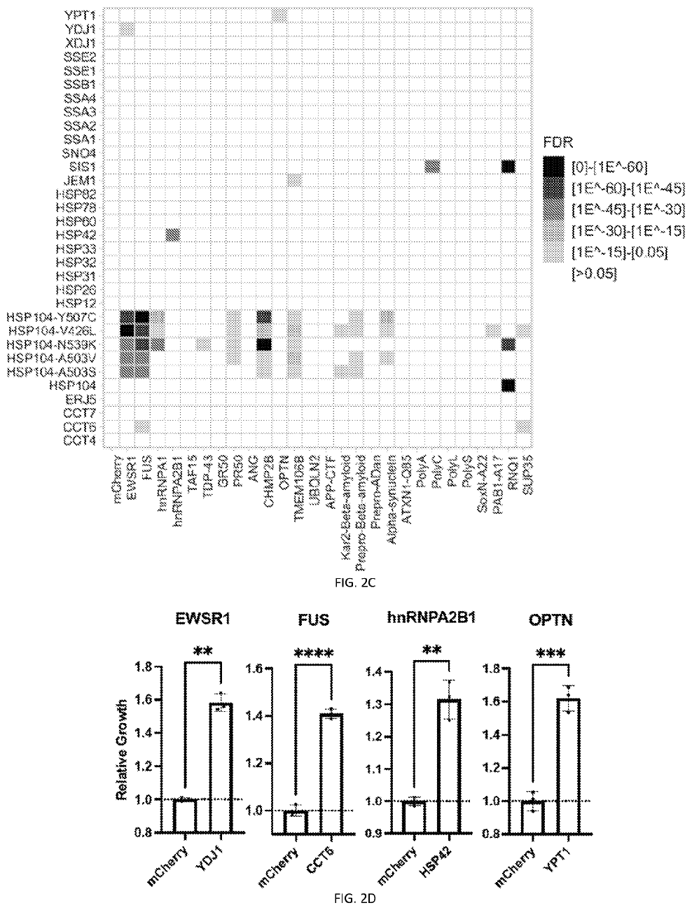

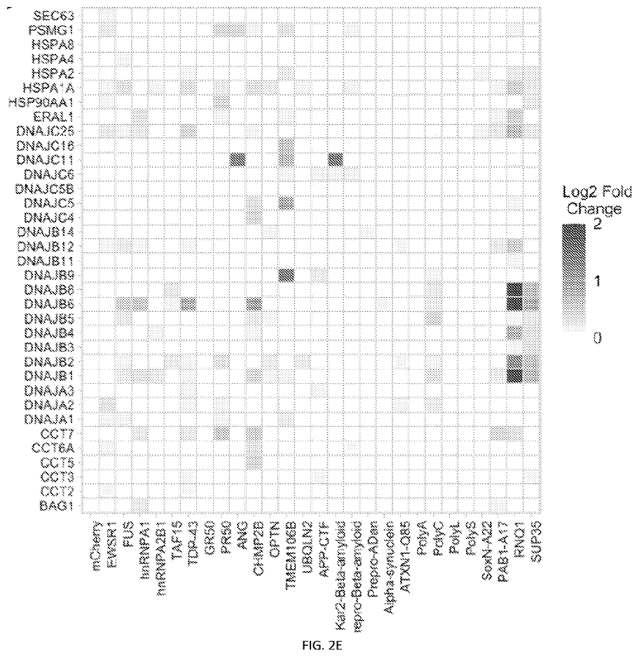

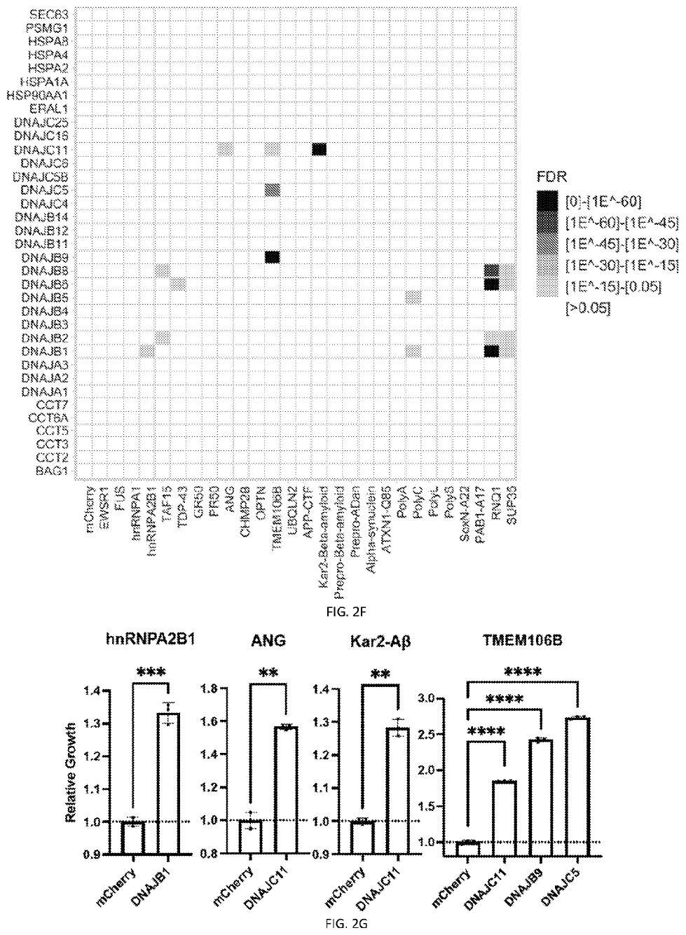

In some embodiments, the aggregation-prone protein is associated with a disease or disorder. In some embodiments, the aggregation-prone protein is associated with a neurodegenerative disease or disorder. In some embodiments, the aggregation-prone protein is selected from those listed in FIG. 2A.

In some embodiments, the putative modifier is a molecular chaperone. Molecular chaperones include proteins that assist (e.g., interact with, stabilize) another protein or protein complex to facilitate the protein or protein complex acquiring or maintaining its correct conformation, while not being present in the final structure or complex. Several different classes of molecular chaperones exist, including stress proteins or heat-shock proteins (HSPs), all of which are suitable for use in the methods and systems disclosed herein. Molecular chaperones, and families thereof, are usually classified according to their molecular weight (e.g., HSP40, HSP60, HSP70, HSP90, HSP100 and the small HSPs).

### Methods

A finding from the disclosed screens was the lack of concordance between models of the same class, such as dipeptide repeat models and poly-alanine models. RNA-binding proteins showed a general trend for having rescuers that were active on several members of the group. No rescuers, however, showed clear activity on all RNA-binding proteins and each tended to show a preference for a particular subset (FIGS. 2, 14, and 15). As such, there may be multiple differing points of shared identity among the class of RNA-binding proteins implicated in NDD such that various genetic modifiers are able to interface with these targets in multiple non-predictable ways to inhibit their proteotoxicity.

The human HSP40 chaperone, DNAJB6, was validated as a rescuer of the proteotoxicity and solubility of FUS, TDP-43, and hnRNPA1. Using FUS as a model client protein, in vitro studies identified a mechanism by which DNAJB6 can prevent aggregation by maintaining phase-separated species in a stable gel-like state. This finding is in contrast to other identified rescuers of FUS misfolding, such as TNPO1, which deters FUS aggregation by dissolving condensates. Extensive deep mutational scanning of DNAJB6 identified enhanced variants.

Disclosed herein are methods for rescuing RNA-binding protein misfolding or preventing RNA-binding protein aggregation comprising contacting the RNA-binding protein with DNAJB6 or an active fragment or variant thereof. In some embodiments, the RNA-binding protein comprises Fused in Sarcoma (FUS), TAR DNA-binding protein 43 (TDP-43), heterogeneous nuclear ribonucleoprotein A1 (hnRNPA1), or a combination thereof.

In some embodiments, the RNA-binding protein is in a cell and the contacting comprises providing DNAJB6 or an active fragment or variant thereof, or a nucleic acid encoding the DNAJB6 or an active fragment or variant thereof to the cell. In some embodiments, the cell is in a subject and the contacting comprises administering DNAJB6 or an active fragment or variant thereof, or a nucleic acid encoding the DNAJB6 or an active fragment or variant thereof to the subject.

Also disclosed herein are methods for treating a disease or disorder characterized by protein misfolding or protein aggregation comprising administering DNAJB6 or an active fragment or variant thereof, or a nucleic acid encoding the DNAJB6 or an active fragment or variant thereof to a subject in need thereof. In some embodiments, the disease or disorder is characterized by protein misfolding or protein aggregation of an RNA-binding protein. In some embodiments, the RNA-binding protein comprises FUS, TDP-43, hnRNPA1, or a combination thereof. In some embodiments, the disease or disorder is characterized by RNA-binding protein aggregation (e.g., FUS, TDP-43, hnRNPA1).

In select embodiments, the disease or disorder is characterized as a TDP-43 or FUS proteinopathy. TDP-43 proteinopathies are characterized by the fact that TDP-43 is a protein that mechanistically links frontotemporal lobar degeneration with ubiquitin-positive inclusions (FTLD-U) with and without motor neuron disease to a variety of neurodegenerative diseases. FUS proteinopathies are less defined but are characterized by inclusions containing FUS, for example as in three different forms of frontotemporal dementia. See for example, Lagier-Tourenne, et al., Human Molecular Genetics, Volume 19, Issue R1, 15 Apr. 2010, Pages R46-R64, incorporated herein by reference in its entirety. In select embodiments, the disease or disorder is characterized by one or more mutations in hnRNPA1. See for example, Beijer, et al., JCI Insight. 2021; 6(14):e148363, incorporated herein by reference in its entirety.

In some embodiments, the disease or disorder is a neurodegenerative disease. A “neurodegenerative disease” or a “NDD” refers to a central nervous system disease characterized by progressive, normally gradual, loss of functional neural tissue. Non-limiting examples of neurodegenerative diseases include Alzheimer's disease, Parkinson's disease, Amyotrophic lateral sclerosis, Friedreich's ataxia, Multiple sclerosis, Huntington's disease, Transmissible spongiform encephalopathy, Charcot-Marie-Tooth disease, Dementia with Lewy bodies, corticobasal degeneration, progressive supranuclear palsy, and Hereditary spastic paraparesis. In some embodiments, the disease or disorder is selected from amyotrophic lateral sclerosis (ALS) and frontotemporal dementia (FTD).

“Amyotrophic lateral sclerosis” or “ALS” denote a progressive neurodegenerative disease that affects upper motor neurons (motor neurons in the brain) and/or lower motor neurons (motor neurons in the spinal cord) and results in motor neuron death. As used herein, the term “ALS” includes all of the classifications of ALS, including, but not limited to, classical ALS (typically affecting both lower and upper motor neurons), Primary Lateral Sclerosis (PLS, typically affecting only the upper motor neurons), Progressive Bulbar Palsy (PBP or Bulbar Onset, a version of ALS that typically begins with difficulties swallowing, chewing and speaking), Progressive Muscular Atrophy (PMA, typically affecting only the lower motor neurons) and familial ALS (a genetic version of ALS).

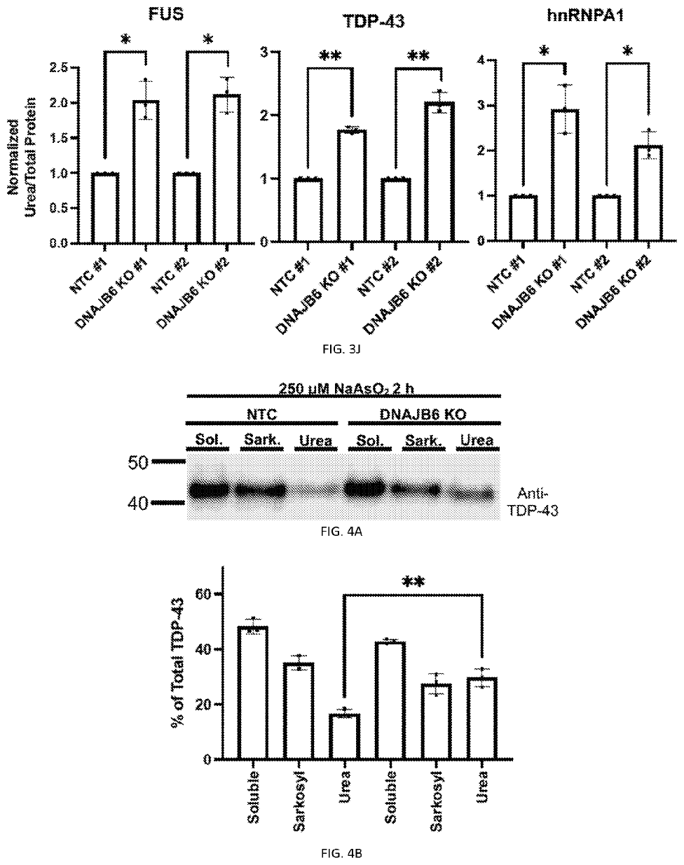

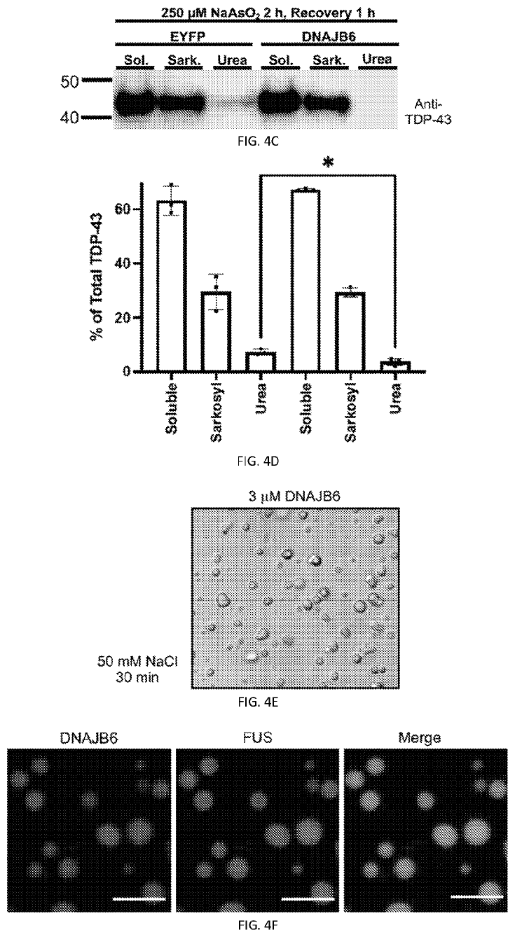

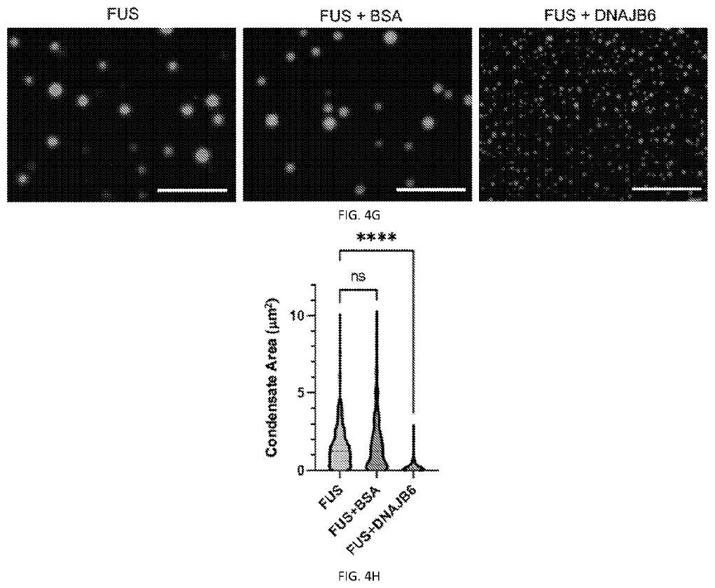

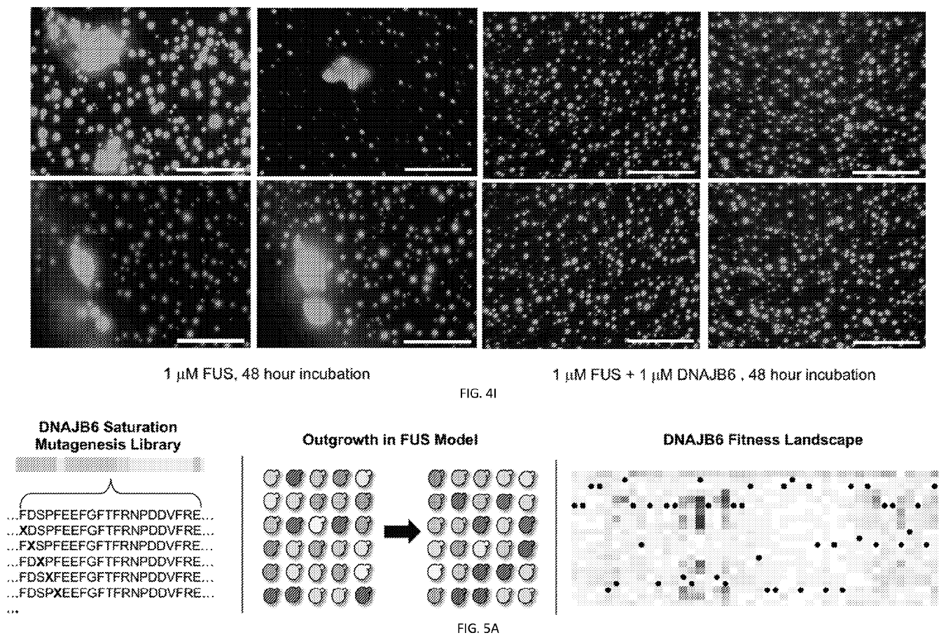

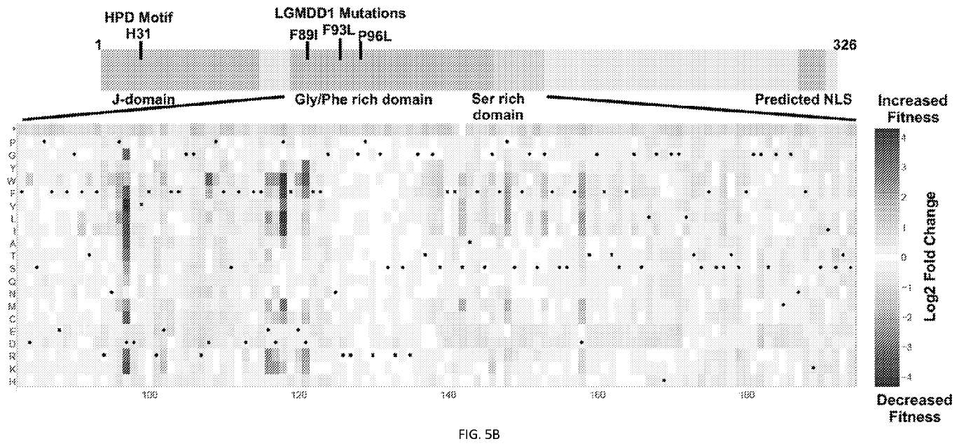

“Frontotemporal dementia,” “FTD,” “frontotemporal degeneration disease,” or “frontotemporal neurocognitive disorder” are used interchangeably herein to denote a group of brain disorders characterized by atrophy of the frontal and/or temporal lobes, subclinical frontal dysfunction, behavior, personality, and language impairment, and progressive aphasia. FTDs may be divided into three primary subtypes, namely behavioral variant, semantic dementia, and progressive non-fluent aphasia. Examples of FTDs include Pick's disease, frontotemporal dementia with motor neuron disease, primary progressive aphasia, and corticobasal degeneration. About half of FTDs are inherited; a higher proportion than most other neurodegenerative diseases. Subjects with FTD may comprise mutations in a gene including, for example, superoxide dismutase 1 gene (SOD1), alsin (ALS2), probably helicase senataxin (SETX), spatacsin (SPG11), fused in sarcoma gene (FUS), vesicle-associated membrane protein-associated protein B/C (VAPB), angiogenin (ANG), transactive response DNA binding protein 43 kDa (TDP-43, TARDBP), polyphosphoinositide phosphatase (FIG. 4), optineurin (OPTN), ataxin-2 (ATXN2), valosin containing protein (VCP), ubiquilin-2 (UBQLN2), sigma-1 receptor (SIGMAR1), charged multivesicular body protein 2B (CHMP2B), profiling-1 (PFN1), receptor tyrosine-protein kinase erbB-4 (ERBB4), heterogeneous nuclear ribonucleoprotein A1 (HNRNPA1), matrin-3 (MATR3), tubulin alpha-4A chain (TUBA4A), C9orf72, coiled-coil-helix-coiled-coil-helix domain containing 10 (CHCHD10), sequestosome-1 (SQSTM1), serine/threonine-protein kinase (TBK1), C9orf72, microtubule associated protein tau (MAPT), progranulin (PGN, GRN), and combinations thereof. Up to 40% of FTD cases have been found to carry a C9orf72 gene mutation, which is also commonly associated with genetic causes of ALS. Several other genes, e.g., TARDBP, SQSTM1, VCP, FUS, TBK1, CHCHD10, can also cause both diseases. FTD with ALS, often referred to as ALS-Frontotemporal spectrum disorder, FTD-ALS, or FTD-motor neuron disease (MND), manifests as both a movement disorder and cognitive or behavioral dementia. As used herein, the term “FTD” includes all of the classifications or etiologies of FTD, including FTD with ALS.

In some embodiments, the methods may further comprise decreasing the expression of disease-related RNA binding proteins such as FUS, TDP-43, or hnRNPA1 (e.g., by the use of antisense oligonucleotides). As such, the methods may further comprise providing to a cell or administering to a subject a transcription or translation inhibitor of a disease-related RNA binding proteins. In some embodiments, the methods comprise providing to a cell or administering to a subject a transcription or translation inhibitor of FUS, TDP-43, and/or hnRNPA1. In select embodiments, the methods comprise providing to a cell or administering to a subject a gene silencing oligonucleotide (e.g., an siRNA, an antisense oligonucleotide, dominant-negative, a short-hairpin RNA, a miRNA, a guideRNA (for example, for use with a gene editing system, e.g., CRISPR/Cas), a dicer-substrate RNA, a DNAzyme, or an aptamer targeting the IGF-1R gene or the IGF-1R messenger RNA), for FUS, TDP-43, and/or hnRNPA1.

In some embodiments, the methods may further comprise co-expressing, administering, or modulating the expression or activity of proteins which interact with DNAJB6, e.g., HSP70 family members. For example, the methods may further comprise providing to a cell or administering to a subject an HSP70 family member protein or a functional fragment or variant thereof, or a nucleic acid encoding the HSP70 family member protein or a functional fragment or variant thereof. Alternatively or additionally, the methods may further comprise administering an agent which increases the amount or intracellular activity of HSP70 in the cell, e.g., an agent which stabilizes an HSP70 family member from degradation, an agent which increases transcription or translation of an HSP70 family member, or an agent with upregulates the heat shock response thereby inducing HSP70 expression (e.g., hydroxylamine derivatives, select anti-inflammatory and anti-neoplastic drugs, sub-lethal heat therapy or hyperthermia, inducers of cellular stress (e.g., reactive oxygen species (ROS), adrenalin, noradrenalin, UV light, radiation therapy).

Heat shock 70 (HSP70) proteins comprise one of the most ubiquitous classes of chaperones assisting in maintaining proper conformation of proteins at any stage of their lifetime. Prokaryotes have three Hsp70 proteins: Dnak, HscA (Hsc66), and HscC (Hsc62). Eukaryotes express several slightly different Hsp70 proteins based on expression patterns and subcellular localization. Eukaryotic HSP70 proteins include, but are not limited to, Hsc70 (Hsp73/HSPA8), Hsp70 (encoded by three very closely related paralogs: HSPA1A, HSPA1B, and HSPA1L), Binding immunoglobulin protein (BiP or Grp78), mtHsp70 or Grp75, HSPA2, HSPA3, HSPA4, HSPA5, HSPA6, HSPA7, HSPA8, HSPA9,

HSP70 proteins often function in concert with cofactors, for example a J protein (also referred to as DnaJ or Hsp40), a nucleotide exchange factor, Hip (Hsp70 interacting protein), and Hop (Hsp-organizing protein). For example, the homo-oligomeric protein Hip (Hsp70 interacting protein) serves as a positive regulator by stabilizing the ADP-bound state of Hsp70 and J-domain and nucleotide exchange factors accelerate ATP hydrolysis and ADP-ATP exchange, respectively. In some embodiments, the methods may further comprise providing any HSP70 cofactor to the cell and/or subject.

In some embodiments, combination therapy of DNAJB6 with decreasing the expression of disease-related RNA binding proteins and/or co-expressing, administering, or modulating the expression or activity of proteins which interact with DNAJB6 (e.g., HSP70 or cofactors thereof) may be administered at the same time or separated by a time interval. For example, combination therapy may be achieved with a single composition or pharmacological formulation that includes DNAJB6 or an active fragment or variant thereof or a nucleic acid encoding the DNAJB6 or an active fragment or variant thereof and HSP70 and/or cofactors thereof or a nucleic acid encoding the HSP70 and/or cofactors thereof and/or a gene silencing oligonucleotide, or with two or more distinct compositions or formulations, administered at the same time or separated by a time interval.

In some embodiments, the methods may further comprise administration with one or more additional therapies to treat the disease or disorder, or one or more symptoms of the disease or disorder. The additional therapy may include administration of an additional therapeutic agent or a therapy not connected to administration of another agent including surgery and physical, occupational, and/or speech therapy. For example, the methods may further comprise one or more additional therapies to treat ALS or FTD or one or more symptoms of ALS or FTD, including, but not limited to, riluzole, edaravone, sodium phenylbutyrate and taurursodiol, cholinesterase inhibitors (e.g., donepezil, rivastigmine, and galantamine), N-methyl-D-aspartic acid (NMDA) receptor antagonists, antidepressants, antipsychotics, physical therapy, occupation therapy, speech therapy, and nutritional support.

The additional therapy may be administered at the same time as the initial therapy. For example, either in the same composition or in a separate composition administered at substantially the same time as the first composition. In some embodiments, the additional therapy may precede or follow the treatment of the initial therapy by time intervals ranging from hours to months.

In some embodiments, the methods described herein decrease proteotoxicity of various cells or tissues. In some embodiments, treatment refers to decreased proteotoxicity due to FUS, TDP-43, or hnRNPA1 in cells (e.g., neuronal or glial cells), brain target tissues, spinal cord neurons, and/or peripheral target tissues. In certain embodiments, proteotoxicity is decreased by about 5%, 10%, 15%, 20%, 25%, 30%, 35%, 40%, 45%, 50%, 55%, 60%, 65%, 70%, 75%, 80%, 85%, 90%, 95%, 97%, 99%, or more as compared to a control. In some embodiments, proteotoxicity is decreased by at least 1-fold, 2-fold, 3-fold, 4-fold, 5-fold, 6-fold, 7-fold, 8-fold, 9-fold, 10-fold, or more as compared to a control. In some embodiments, proteotoxicity is measured by tests known to those of ordinary skill in the art including, but not limited to, neuroimaging methods (e.g., CT scans, MRI, functional MRI, etc.).

In certain embodiments, treatment according to the present disclosure results in a reduction (e.g., about a 5%, 10%, 15%, 20%, 25%, 30%, 40%, 50%, 55%, 60%, 65%, 70%, 75%, 80%, 90%, 95%, 97.5%, 99%, or more reduction) or a complete elimination of the presence, or alternatively the accumulation, of one or more pathological, clinical, or biological markers that are associated with a neurological disease (e.g., FTD and/or ALS). For example, in some embodiments, upon administration to a subject, a pharmaceutical composition described herein demonstrates or achieves a reduction in muscle loss, muscle twitching, muscle weakness, spasticity, abnormal tendon reflexes, Babinski sign, breathing problems, facial weakness, slurred speech, loss of perception, loss of reasoning, loss of judgment, and/or loss of imagination.

In some embodiments, treatment refers to increased survival (e.g., survival time). For example, treatment can result in an increased life expectancy of a patient. In some embodiments, treatment results in an increased life survival by more than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150%, about 155%, about 160%, about 165%, about 170%, about 175%, about 180%, about 185%, about 190%, about 195%, about 200% or more, as compared to an average survival time (e.g., life expectancy of one or more control individuals with a select neurodegenerative disease without treatment). In some embodiments, treatment results in an increased life expectancy of a patient by more than about 6 months, about 7 months, about 8 months, about 9 months, about 10 months, about 11 months, about 12 months, about 2 years, about 3 years, about 4 years, about 5 years, about 6 years, about 7 years, about 8 years, about 9 years, about 10 years or more, as compared to the average life expectancy of one or more control individuals without treatment. In some embodiments, treatment results in long term survival of a patient. As used herein, the term “long term survival” refers to a survival time or life expectancy longer than about 40 years, 45 years, 50 years, 55 years, 60 years, or longer.

The term “improve,” “increase” or “reduce,” as used herein, indicates values that are relative to a control. In some embodiments, a suitable control is a baseline measurement, such as a measurement in the same cell or same individual prior to initiation of the treatment described herein, or a measurement in a control cell(s) or individual(s) in the absence of the treatment described herein. For example, a “control individual” is an individual afflicted with a select neurodegenerative disease, who is approximately the same age and/or gender as the individual being treated (to approximate that the stages of the disease in the treated individual and the control individual(s) are comparable).

### a) DNAJB6

In some embodiments, the DNAJB6 comprises a sequence corresponding to, or substantially corresponding to, the wild-type version of the protein. For example, the sequence may substantially correspond to the wild-type protein sequence except for changes made for facile cloning or removal of known restriction sites. Thus, protein products from potential alternative start codons compared to the predicted nucleic acid sequences in this document are therefore not excluded.

In some embodiments, the DNAJB6 comprises an amino acid sequence having at least 70% (e.g., at least 75%, at least 80%, at least 85%, at least 90%, at least 95%, at least 97%, at least 98%, at least 99%) identity to SEQ ID NO: 1 or SEQ ID NO: 12, corresponding to DNAJB6 isoforms a and b, respectively.

Variants of DNAJB6 may comprise one or more amino acid substitutions as compared to the wild-type sequence, SEQ ID NO: 1, or SEQ ID NO: 12. An amino acid “replacement” or “substitution” refers to the replacement of one amino acid at a given position or residue by another amino acid at the same position or residue within a polypeptide sequence. Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or He), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gin), lysine (K or Lys), and arginine (R or Arg).

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra). Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH₂ can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

In some embodiments, the DNAJB6 variants have increased activity for rescuing FUS-mediated toxicity, as described herein. In some embodiments, the DNAJB6 variant comprises one or more mutations or substitutions in an acidic residue between amino acids 138-171 and/or 182-189, as compared to the wild-type sequence, SEQ ID NO:1, or SEQ ID NO: 12. In some embodiments, the DNAJB6 variant comprises one or more mutations or substitutions at positions 101, 107, 140, 160, 171, 182, and 192, as compared to the wild-type sequence, SEQ ID NO:1, or SEQ ID NO: 12. In some embodiments, the DNAJB6 variant comprises one or more mutations or substitutions of R101, R107, F140, G160, G171, G182, and S192, as compared to the wild-type sequence, SEQ ID NO: 1, or SEQ ID NO: 12. In some embodiments, the DNAJB6 variant comprises one or more mutations or substitutions of G182 and S192, as compared to the wild-type sequence, SEQ ID NO: 1, or SEQ ID NO: 12. In some embodiments, the DNAJB6 variant comprises one or more mutations or substitutions selected from the group consisting of R101E, R107D, F140D, G160D, G171D, G182E, and S192T, as compared to the wild-type sequence, SEQ ID NO:1, or SEQ ID NO: 12. In select embodiments, DNAJB6 variant comprises G182E and S192T mutations or substitutions, as compared to the wild-type sequence, SEQ ID NO: 1, or SEQ ID NO: 12.

The application also provides polypeptides comprising the DNAJB6 variants, compositions comprising the DNAJB variants, and nucleic acids encoding thereof. For example, provided herein are polypeptides comprising a DNAJB6 variant comprising an amino acid sequence having one or more substitutions in an acidic residue between amino acids 138-171 and/or 182-189 relative to SEQ ID NO: 1 or SEQ ID NO: 12. In some embodiments, the polypeptide comprises an amino acid sequence having one or more mutations or substitutions at positions 101, 107, 140, 160, 171, 182, and 192 relative to SEQ ID NO: 1 or SEQ ID NO: 12. In some embodiments, the polypeptide comprises an amino acid sequence having one or more mutations or substitutions selected from the group consisting of: R101E, R107D, F140D, G160D, G171D, G182E, and S192T. In some embodiments, the polypeptide comprises mutations or substitutions at positions 182 and 192 relative to SEQ ID NO: 1 or SEQ ID NO: 12. In some embodiments, the polypeptide comprises mutations or substitutions of G182E and S192T relative to SEQ ID NO: 1 or SEQ ID NO: 12.

Alternatively, or in addition to, the disclosed methods, the activity or expression of DNAJB6 may be modulated by other means aside from administering exogenous amounts of DNAJB6 or a variant thereof, or a nucleic acid encoding the DNAJB6 or a variant thereof. For example, methods of tissue selective regulation of its expression or activity may be used, e.g., CRISPR targeting to either alter its amino acid composition or introduce changes into its promoter to modulate its expression profile. Other approaches such as small molecule regulators of its expression or activity can also be explored, as can other orthogonal methods of regulating gene expression and RNA sequence such as antisense oligonucleotides or approaches that directly bind to RNA and recruit secondary effectors such as RNA modification machinery.

### b) Administration

The proteins (e.g., DNAJB6 or an active fragment or variant thereof, HSP70 family member proteins and cofactors thereof) and nucleic acids (e.g., nucleic acids and vectors encoding the disclosed proteins, gene silencing oligonucleotides) disclosed herein may be administered to a subject by a variety of methods. In any of the uses or methods described herein, administration may be by various routes known to those skilled in the art, including without limitation oral, inhalation, intravenous, intramuscular, topical, subcutaneous, systemic, and/or intraperitoneal administration to a subject in need thereof.

The methods may comprise administering to the subject, in vivo, or by transplantation of ex vivo treated cells, a therapeutically effective amount of DNAJB6 or an active fragment or variant thereof, or a nucleic acid encoding the DNAJB6 or an active fragment or variant thereof.

DNAJB6 or an active fragment or variant thereof, HSP70 family members, or a nucleic acid encoding the DNAJB6 or an active fragment or variant thereof or ex vivo treated cells may be administered with a pharmaceutically acceptable carrier or excipient as a pharmaceutical composition. In some embodiments, DNAJB6 or an active fragment or variant thereof, or a nucleic acid encoding the DNAJB6 or an active fragment or variant thereof may be mixed with a pharmaceutically acceptable carrier to form pharmaceutical compositions, which are also within the scope of the present disclosure.

In some embodiments, the DNAJB6 or an active fragment or variant thereof, or a nucleic acid encoding the DNAJB6 or an active fragment or variant thereof is in a pharmaceutical composition with one or more of a HSP70 family member protein or a cofactor thereof, or a nucleic acid encoding the HSP70 family member protein or cofactor thereof. In some embodiments, the DNAJB6 or an active fragment or variant thereof, or a nucleic acid encoding the DNAJB6 or an active fragment or variant thereof is in a pharmaceutical composition with a gene silencing oligonucleotide, as disclosed herein.

In some embodiments, an effective amount of the DNAJB6 or an active fragment or variant thereof, or a nucleic acid encoding the DNAJB6 or an active fragment or variant thereof or a composition comprising thereof can be administered. As used herein the term “effective amount” may be used interchangeably with the term “therapeutically effective amount” and refers to that quantity that is sufficient to result in a desired activity upon administration to a subject in need thereof. Within the context of the present disclosure, the term “effective amount” refers to that quantity of the components of the system such that proteotoxicity and/or aggregation is decreased and solubility of FUS, TDP-43, and hnRNPA1 is increased.

When utilized as a method of treatment, the effective amount may depend on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human.

In the context of the present disclosure insofar as it relates to any of the disease conditions recited herein, the terms “treat,” “treatment,” and the like mean to relieve or alleviate at least one symptom associated with such condition, or to slow or reverse the progression of such condition. Within the meaning of the present disclosure, the term “treat” also denotes to arrest, delay the onset (e.g., the period prior to clinical manifestation of a disease) and/or reduce the risk of developing or worsening a disease,

The phrase “pharmaceutically acceptable,” as used in connection with compositions and/or cells of the present disclosure, refers to molecular entities and other ingredients of such compositions that are physiologically tolerable and do not typically produce untoward reactions when administered to a subject (e.g., a mammal, a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g., Remington: The Science and Practice of Pharmacy 20th Ed. (2000) Lippincott Williams and Wilkins, Ed. K. E. Hoover.

### c) Nucleic Acids

The present disclosure also provides for DNA segments encoding the proteins (e.g., DNAJB6 or an active fragment or variant thereof, HSP70 family member proteins and cofactors thereof) and nucleic acids (e.g., nucleic acids and vectors encoding the disclosed proteins, gene silencing oligonucleotides) disclosed herein, vectors containing these segments and cells containing the vectors. The vectors may be used to propagate the segment in an appropriate cell and/or to allow expression from the segment (e.g., an expression vector). The person of ordinary skill in the art would be aware of the various vectors available for propagation and expression of a nucleic acid sequence.

The nucleic acid encoding the proteins (e.g., DNAJB6 or an active fragment or variant thereof, HSP70 family member proteins and cofactors thereof) disclosed herein may be any nucleic acid including DNA, RNA, or combinations thereof. In some embodiments, the nucleic acid encoding the proteins (e.g., DNAJB6 or an active fragment or variant thereof, HSP70 family member proteins and cofactors thereof) comprises a messenger RNA or a vector.

In certain embodiments, engineering the nucleic acid for use in eukaryotic cells may involve codon-optimization. It will be appreciated that changing native codons to those most frequently used in mammals allows for maximum expression of the system proteins in mammalian cells (e.g., human cells). Such modified nucleic acid sequences are commonly described in the art as “codon-optimized,” or as utilizing “mammalian-preferred” or “human-preferred” codons. In some embodiments, the nucleic acid sequence is considered codon-optimized if at least about 60% (e.g., 65%, 70%, 75%, 80%, 85%, 90%, 95%, or 98%) of the codons encoded therein are mammalian preferred codons.

The present disclosure further provides engineered, non-naturally occurring vectors and vector systems, which can encode the proteins (e.g., DNAJB6 or an active fragment or variant thereof, HSP70 family member proteins and cofactors thereof) and nucleic acids (e.g., nucleic acids and vectors encoding the disclosed proteins, gene silencing oligonucleotides) disclosed herein. The vector(s) can be introduced into a cell that is capable of expressing the polypeptide encoded thereby, including any suitable prokaryotic or eukaryotic cell.

The vectors of the present disclosure may be delivered to a eukaryotic cell in a subject. Modification of the eukaryotic cells can take place in a cell culture, where the method comprises isolating the eukaryotic cell from a subject prior to the modification. In some embodiments, the method further comprises returning said eukaryotic cell and/or cells derived therefrom to the subject.

Viral and non-viral based gene transfer methods can be used to introduce nucleic acids encoding the proteins (e.g., DNAJB6 or an active fragment or variant thereof, HSP70 family member proteins and cofactors thereof) and nucleic acids (e.g., nucleic acids and vectors encoding the disclosed proteins, gene silencing oligonucleotides) disclosed herein into cells, tissues, or a subject. Such methods can be used to administer nucleic acids encoding the proteins (e.g., DNAJB6 or an active fragment or variant thereof, HSP70 family member proteins and cofactors thereof) and nucleic acids (e.g., nucleic acids and vectors encoding the disclosed proteins, gene silencing oligonucleotides) disclosed herein to cells in culture, or in a host organism. Non-viral vector delivery systems include DNA plasmids, cosmids, RNA (e.g., a transcript of a vector described herein), a nucleic acid, and a nucleic acid complexed with a delivery vehicle. Viral vector delivery systems include DNA and RNA viruses, which have either episomal or integrated genomes after delivery to the cell. Viral vectors include, for example, retroviral, lentiviral, adenoviral, adeno-associated and herpes simplex viral vectors.

In certain embodiments, plasmids that are non-replicative, or plasmids that can be cured by high temperature may be used, such that the nucleic acid encoding the proteins (e.g., DNAJB6 or an active fragment or variant thereof, HSP70 family member proteins and cofactors thereof) and nucleic acids (e.g., nucleic acids and vectors encoding the disclosed proteins, gene silencing oligonucleotides) disclosed herein may be removed from the cells under certain conditions.

A variety of viral constructs may be used to deliver the proteins (e.g., DNAJB6 or an active fragment or variant thereof, HSP70 family member proteins and cofactors thereof) and nucleic acids (e.g., nucleic acids and vectors encoding the disclosed proteins, gene silencing oligonucleotides) disclosed herein to the targeted cells and/or a subject. Nonlimiting examples of such recombinant viruses include recombinant adeno-associated virus (AAV), recombinant adenoviruses, recombinant lentiviruses, recombinant retroviruses, recombinant herpes simplex viruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7(1):33-40; and Walther W. and Stein U., 2000 Drugs, 60(2): 249-71, incorporated herein by reference.

In one embodiment, a DNA segment encoding the proteins (e.g., DNAJB6 or an active fragment or variant thereof, HSP70 family member proteins and cofactors thereof) and nucleic acids (e.g., nucleic acids and vectors encoding the disclosed proteins, gene silencing oligonucleotides) disclosed herein is contained in a plasmid vector that allows expression of the protein(s) and subsequent isolation and purification of the protein produced by the recombinant vector. Accordingly, the proteins can be purified following expression, obtained by chemical synthesis, or obtained by recombinant methods.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

Vectors of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EFla (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Moreover, inducible and tissue specific expression of an RNA or protein can be accomplished by placing the nucleic acid encoding such a molecule under the control of an inducible or tissue specific promoter/regulatory sequence. Examples of tissue specific or inducible promoter/regulatory sequences which are useful for this purpose include, but are not limited to, the rhodopsin promoter, the MMTV LTR inducible promoter, the SV40 late enhancer/promoter, synapsin 1 promoter, ET hepatocyte promoter, GS glutamine synthase promoter and many others. Various ubiquitous as well as tissue-specific promoters and tumor-specific are commercially available, for example from InvivoGen. In addition, promoters which are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein or RNA operably linked thereto.

The vectors of the present disclosure may direct expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; 5′- and 3′-untranslated regions for mRNA stability and translation efficiency from highly-expressed genes like α-globin or β-globin; SV40 polyoma origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the chimeric receptor. Suitable vectors and methods for producing vectors containing transgenes are well known and available in the art. Selectable markers also include chloramphenicol resistance, tetracycline resistance, spectinomycin resistance, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRP1 genes of S. cerevisiae.

When introduced into the cell, the vectors may be maintained as an autonomously replicating sequence or extrachromosomal element or may be integrated into host DNA.

The present proteins, nucleic acids encoding these proteins, and compositions comprising the proteins and/or nucleic acids described herein may be delivered by any suitable means. In certain embodiments, they are delivered in vivo, as described above. In other embodiments, they are delivered to isolated/cultured cells (e.g., autologous iPS cells) in vitro (e.g., to provide modified cells useful for in vivo delivery to patients afflicted with a disease or condition).

Vectors according to the present disclosure can be transformed, transfected, or otherwise introduced into a wide variety of host cells. Transfection refers to the taking up of a vector by a host cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Any of the vectors comprising a nucleic acid sequence that encodes proteins (e.g., DNAJB6 or an active fragment or variant thereof, HSP70 family member proteins and cofactors thereof) and nucleic acids (e.g., nucleic acids and vectors encoding the disclosed proteins, gene silencing oligonucleotides) disclosed herein is also within the scope of the present disclosure. Such a vector may be delivered into host cells by a suitable method. Methods of delivering vectors to cells are well known in the art and may include DNA or RNA electroporation, transfection reagents such as liposomes or nanoparticles to delivery DNA or RNA; delivery of DNA, RNA, or protein by mechanical deformation (see, e.g., Sharei et al. Proc. Natl. Acad. Sci. USA (2013) 110(6): 2082-2087, incorporated herein by reference); or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the vector can be delivered by any method appropriate for introducing nucleic acids into a cell.

Additionally, delivery vehicles such as nanoparticle-and lipid-based mRNA or protein delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1: 27) and Ibraheem et al. (Int J Pharm. 2014 Jan. 1;459(1-2): 70-83), incorporated herein by reference.

### Kits/Systems

In another aspect, the disclosure provides kits or systems comprising a DNAJB6 or an active fragment or variant thereof, a nucleic acid encoding the DNAJB6 or an active fragment or variant thereof, or a composition thereof, and instructions for using the protein, nucleic acid, or composition.

The kits or systems can also comprise other agents and/or products co-packaged, co-formulated, and/or co-delivered with other components. For example, the kits or systems may further comprise proteins which interact with DNAJB6 (e.g., HSP70 or cofactors thereof) or nucleic acid encoding thereof, gene silencing oligonucleotides or nucleic acid encoding thereof, and/or one or more additional therapies to treat ALS or FTD or one or more symptoms of ALS or FTD.

The kits can also comprise instructions for using the components of the kit. The instructions are relevant materials or methodologies pertaining to the kit. The materials may include any combination of the following: background information, list of components, brief or detailed protocols for using the compositions, trouble-shooting, references, technical support, and any other related documents. Instructions can be supplied with the kit or as a separate member component, either as a paper form or an electronic form which may be supplied on computer readable memory device or downloaded from an internet website, or as recorded presentation.

The kits provided herein are in suitable packaging. Suitable packaging includes, but is not limited to, vials, bottles, jars, flexible packaging, and the like. Individual member components of the kits may be physically packaged together or separately.

It is understood that the disclosed kits or systems can be employed in connection with the disclosed methods. The kits or systems may further contain containers or devices for use with the methods or compositions disclosed herein, for example delivery devices (e.g., syringes and the like).

### EXAMPLES

The following are examples of the present invention and are not to be construed as limiting.

### Materials and Methods

Yeast Strains and Media Barcoded S. cerevisiae yeast BY4741 MATa his3Δ1 leu2Δ0 ura3Δ0 met15Δ0 strains were purchased from Horizon (Cat. #YSC5117). To introduce rescuers through mating, rescuer containing S. cerevisiae yeast BY4742 strains MATa his3Δ1 leu2Δ0 ura3Δ0 lys2Δ0 were used. Individual barcoded strains containing expression vectors were maintained in Synthetic Complete (SC)-ura media (20 g/L glucose, 1.5 g/L Drop Out mix [US Biological D0539-09A], 1.7 g/L Yeast Nitrogen Base [US Biological Y2030], 5 g/L Ammonium Sulfate [Fisher H8N2O45], supplemented with 18 mg/L Leucine and 9 mg/L Histidine). Individual rescuer BY4742 strains were maintained in SC-his media (20 g/L glucose, 1.5 g/L Drop Out mix [US Biological D0539-09A], 1.7 g/L Yeast Nitrogen Base [US Biological Y2030], 5 g/L Ammonium Sulfate [Fisher H8N2O45], supplemented with 18 mg/L Leucine and 1.8 mg/L Uracil). Mating was conducted in YPD media (20 g/L glucose, 20 g/L peptone, and 10 g/L yeast extract). Selection for mated strains was conducted in SC-ura-his media (20 g/L glucose, 1.5 g/L Drop Out mix [US Biological D0539-09A], 1.7 g/L Yeast Nitrogen Base [US Biological Y2030], 5 g/L Ammonium Sulfate [Fisher H8N2O45], supplemented with 18 mg/L Leucine), while outgrowth of induced mated strains was carried out in SC-ura-his gal media (20 g/L galactose, 1.5 g/L Drop Out mix [US Biological D0539-09A], 1.7 g/L Yeast Nitrogen Base [US Biological Y2030], 5 g/L Ammonium Sulfate [Fisher H8N2O45], supplemented with 18 mg/L Leucine).

Plasmids Proteotoxic genes and controls were cloned into either the pAG416GAL-ccdb (Addgene #14147) or pAG426GAL-ccdb (Addgene #14155) using Gateway LR II Clonase Enzyme mix (Invitrogen). Once expression plasmids were sequence verified, they were transformed into barcoded BY4741 strains using standard LiOAc transformation protocols and plated on SC-ura agar plates. Yeast rescuer genes and control rescuer genes were cloned into pAG413GAL-ccdb (addgene #14141) using Gateway cloning. Human rescuer genes from the hOrfeome V8.1 Library collection were cloned into a derivative of pAG413GAL-ccdb, pAG413GAL-ccdb-6Stop, wherein the 3′ attR2 site was modified to encode a stop codon 6 amino acids downstream of the last codon to compensate for a lack of a stop codon in the ORFeome.

All mammalian expression vectors were cloned into the pLEX307 backbone (Addgene #41392) using Gateway LR II Clonase Enzyme mix (Invitrogen).

Plasmid DNA was isolated using standard miniprep buffers (Omega Biotek) and silica membrane columns (Biobasic). All expression plasmids were Sanger sequenced to confirm the appropriate insert (Genewiz).

Yeast Multiplexed Screening Each plate of rescuers was screened in biological duplicates. A fresh aliquot (500 μL) of frozen barcoded yeast pool was inoculated into 5 mL of SC-ura media and rotated at 30° C. At the same time, 5 μL of each rescuer strain was inoculated into 500 μL of SC-his media in 96 well 2 mL deep well plate format (VWR) and shaken at 900 rpm at 30° C. 24 h later, 5 μL of the saturated barcoded yeast pool was mixed individually with 5 μL of rescuer strain in a new 96 well plate where each well was filled with 500 μL of YPD and shaken at 900 rpm at 30° C. For selection of mated strains, 20 h later, 5 μL of mated barcoded yeast pool was transferred into a new 2 mL deep well plate filled with 500 μL of SC-ura-his media and shaken at 900 rpm at 30° C. for 24 h. For outgrowth, 2 μL of the mated and selected pool was inoculated into 1 mL of SC-ura-his galactose media and shaken at 1,000 rpm at 30° C. for 30 h.

After growth, 100 μL of yeast culture was removed and the optical density (OD595) of the culture was determined in a 96 well plate reader (Tecan). After measurement of culture density, genomic DNA was extracted using a modified LiOAc-SDS extraction method. Briefly, plates were centrifuged for 5 min at 4,000 rpm. Supernatant was discarded and the pellet was resuspended in 200 μL of 200 mM LiOAc with 1% SDS with rigorous pipetting. Plates were sealed with aluminum foil and incubated at 70° C. for 20 min to enable lysis. 600 μL 100% ethanol was added to each well and pipetted up and down rigorously before being centrifuged for 10 min at 4,000 rpm. Supernatant was discarded and pellets were air dried for 30 min under flame. Pellets were then resuspended in 200 μL 1× TE and incubated at 42° C. for 30 min. The plates were centrifuged for 10 min at 4,000 rpm and the supernatant containing DNA was pipetted into a new plate for storage at −20° C. Raw sequencing reads from the chaperone screen have been uploaded to the NCBI SRA under BioProject PRJNA769721 (SUB10508463). Raw sequencing reads from the orfeome screen have been uploaded to the NCBI SRA under BioProject PRJNA769721 (SUB10508562).

Sequencing Library Preparation For sequencing on NextSeq 500/550 (Illumina), libraries were prepared from genomic DNA in two PCR steps. The first step amplifies genomic DNA containing the DNA barcode and attaches an internal index to designate which column the well was amplified from. The second PCR attaches Illumina indexes to the amplicon, wherein the combination of Illumina indexes indicates the row and plate location of the well. The first PCR step was done in technical duplicates unless otherwise stated with Taq polymerase (Enzymatics). The following reaction mix was used: 2 μL 10× Taq buffer, 0.1 μL 100 μM forward primer, 0.1 μL 100 μM reverse primer, 0.1 μL Taq polymerase, 0.4 μL 10 mM dNTPs, 0.5 μL DNA, and 16.8 μL H2O. The following cycling conditions were used: 1. 94° C., 180 s, 2. 94° C., 30 s, 3. 60° C., 20 s, 4. 72° C., 30 s, 5. Return to step 2 27×, 6. 72° C., 180 s. After the first round of PCR, technical replicates of each individual well were pooled. For the second round of PCR where Illumina indexes were attached, the following reaction mix was used: 2 μL 10× Taq buffer, 0.1 μL 100 μM forward primer, 0.1 μL 100 μM reverse primer, 0.1 μL Taq polymerase, 0.4 μL 10 mM dNTPs, 0.5 μL DNA from first round PCR, and 16.8 μL H2O. The following cycling conditions were used: 1. 94° C., 180 s, 2. 94° C., 30 s, 3. 56° C., 20 s, 4. 72° C., 30 s, 5. Return to step 2 7×, 6. 72° C., 180 s. After the second round PCR, all reactions corresponding to a plate of screening were pooled together. The reaction products were run out on a gel and a band corresponding to the right size was gel extracted. Libraries were quantified with the NEBNext Library Quant Kit for Illumina according to manufacturer instructions (NEB). Pooled libraries were combined and sequenced with a 75 cycles NextSeq 500/550 High Output Kit on a NextSeq 500/550 machine (Illumina).

Analysis of Multiplexed Screening Raw reads in fastq format were trimmed and assigned to wells via combinations of Illumina indexes and column designating internal indexes. 20 bp barcode sequences were aligned to a reference genome allowing for +1 or −1 shifts in the sequencing phase using bowtie2. Raw counts of exact matches for each barcode were determined and well-read counts were normalized by the total number of reads in that well and converted to counts per million (CPM) unless otherwise stated. Wells were analyzed in batches with other wells in the same plate. Wells with less than 15,000 total reads were discarded in addition to wells where 1 biological replicated received less than 15,000 total reads. After CPM normalization, the estimated actual abundance of reads was calculated by normalizing against the optical density (OD595) of that well, which was measured immediately prior to harvesting with a 96 well Infinite F50 plate reader (Tecan). Wells containing control or inert rescuers were identified and the average read counts of each barcode in controls was determined. The variance in the number of reads between control wells for each barcode was determined. The mean-variance relationship was modeled using the equation log(σvariance-mean)=log(k)+b*log(mean)σ. The barcode mean and adjusted variance were used to determine whether a barcode in a test well was significantly upregulated using a one-sided cumulative density function assuming a normal distribution. The associated p-value was adjusted using a Benjamini-Hochberg procedure correcting for the number of tests in that well. To obtain model level information from individual barcode strains, p-values from independent barcode strains associated with the same model were combined using Stouffer's method. After this summary value was obtained, a further Benjamini-Hochberg procedure was used to correct summary values for the number of models and the number of wells in each plate. Average log2 fold change was calculated as the base 2 logarithm of the average change in counts over the expected value in the control wells. Analysis was conducted with custom scripts in R Version 4.0.2.

Spot Assays Yeast strains to be assayed were grown overnight in selective synthetic complete media until saturation was reached. Saturated cultures were serially diluted 1:5 in sterile PBS (Gibco). To SC-ura-his glucose or galactose agar plates, 5 μL of diluted culture was spotted. Plates were left for 30 minutes to dry before being inverted and incubated at 30° C. for 48 h, followed by being scanned to document growth.

Yeast Liquid Culture Growth Assay Proteotoxic yeast strains were grown in 500 μL SC-ura media in plate format shaken at 1,000 rpm at 30° C. for 24 h. At the same time, rescuer yeast strains were grown in 500 μL SC-his media shaken at 1,000 rpm at 30° C. for 24 h. After growth, 5 μL of appropriate proteotoxic and rescuer yeast strains were mixed in 500 μL YPD and shaken at 1,000 rpm at 30° C. for 24 h. To 500 μL of dual selective SC-ura-his media, 5 μL of mated strains were inoculated and shaken at 1,000 rpm at 30° C. for 30 h. 48 h prior to reading, mated and selected strains were inoculated in SC-ura-his galactose media at one of 3 dilution factors depending on their growth rate and shaken at 1,000 rpm at 30° C. for 24 h. 24 h after initial inoculation, strains were passaged depending on their specified dilution factor into fresh SC-ura -his galactose media. Upon reaching the assay endpoint, 100 μL of each well was transferred to a 96 well plate (Greiner) and the optical density was determined on a 96 well plate reader (Tecan). Multiple media only wells were also quantified as a baseline and these values were subtracted from optical density measurements. All statistics were performed in GraphPad Prism Version 9.2.0.

Protein Harvesting and Western Blotting RIPA Urea Extractions: Cells in 24-well dishes were washed with ice cold PBS (Gibco) after media was removed. 250 μL of RIPA buffer (50 mM Tris-HCl (pH 7.4), 150 mM NaCl 1% NP-40, 0.5% sodium deoxycholate and 0.1% SDS, Alfa Aesar) was added to each well and allowed to sit for 2 min. Cells were resuspended in RIPA buffer and moved to conical tubes. To lyse cells further, cells were sonicated for 10 s while kept on ice. Cell lysate was centrifuged for 20 min at 12,000×g at 4° C. and supernatant was saved as RIPA soluble fraction. Pellets were washed 1× with RIPA buffer and 50 μL urea buffer was added (8M Urea, 2M Thiourea, 4% CHAPS,) with vigorous pipetting to resuspend pellet and spun for 20 min at 12,000×g at 4° C. The concentration of protein in the RIPA buffer was determined with a Bradford assay and lysates were adjusted to a final concentration between 250-350 μg/μL depending on the yield of the lowest concentration of the lysate in the set in which it was processed with 1× LDS loading buffer (Invitrogen). RIPA soluble fractions were boiled for 5 minutes and stored at −80° C. until use. Urea fractions were stored at −80° C. without boiling.

Soluble/Sarkosyl/Urea Extractions: Cells in 6-well dishes were washed with ice cold PBS (Gibco) after media was removed. 500 μL of soluble buffer (0.1 M MES pH 7.0, 1 mM EDTA, 0.5 mM MgSO4, 1 M sucrose) was added to cells and cells were scraped off the plate and transferred into Eppendorf tubes. To lyse cells, cells were sonicated for 40 s in 10 s intervals on ice. Cell lysate was centrifuged for 30 min at 20,000×g at 4° C. Supernatant was removed and saved as the soluble fraction. Pellets were washed with 200 μL of soluble buffer before being resuspended in 250 μL of Sarkosyl buffer (0.1 MES pH 6.8, 10% sucrose, 2 mM EGTA, 0.5 mM MgSO4, 500 mM NaCl, 1 mM MgCl2, 10 mM NaH2PO4, 20 mM NaF, 1% N-lauroylsarcosine). The mixture was subject to end-over-end rotation overnight at 4° C. The next day, samples were centrifuged for 100,000×g at 4° C. for 40 min. Supernatant was removed and saved as the sarkosyl soluble fraction. Pellets were washed with 200 μL of sarkosyl buffer before being resuspended in 125 μL of urea buffer. The concentration of protein in the soluble buffer was determined with a Bradford assay and lysates were adjusted to a final concentration between 250-500 μg/μL depending on the yield of the lowest concentration of the lysate in the set in which it was processed with 1× LDS loading buffer (Invitrogen). Soluble and sarkosyl fractions were boiled for 5 minutes and stored at −80° C. until use. Urea fractions were stored at −80° C. without boiling.

Yeast Lysate Extraction: Cells were collected and centrifuged at 2,300×g for 2 min and washed with 1 mL of dH2O. To pelleted yeast cells, 200 μL of 0.1M NaOH was added and cells were resuspended by vortexing. Cells were allowed to lyse for 10 min at room temperature. Lysed cells were spun at 13,000×g for 1 min and supernatant was discarded. Pellets were resuspended in 50 μL of dH2O and 25 μL of 200 mM DTT (Fisher) was added along with 25 μL of 4× LDS loading buffer (Invitrogen). Samples were boiled at 95° C. for 5 min and subsequently centrifuged at 800×g for 10 min at 4° C. Supernatants were collected and moved to a new tube for storage at −20° C.

Western Blotting: 10 μL of normalized lysate with loading buffer was loaded into NuPAGE 4 to 12% Bis-Tris protein gels (Invitrogen) and subjected to 100 V electrophoresis for 65 min. Separated proteins were transferred onto a 0.2 μM PVDF membrane and blocked with SuperBlock (Invitrogen). Primary antibodies were diluted in SuperBlock with 0.1% Tween-20 and incubated overnight at 4° C. with gentle rotation. Blots were washed with TBST before secondary antibody incubation. Blots were imaged with the Odyssey XF imaging system (Li-Cor) using the chemiluminescent detection. After transfer, blots were stained with Ponceau S stain (G Biosciences) for 15 min. Total protein was imaged on a LAS-4000 imager (Fujifilm). Band intensities were quantified with Image Studio Lite (Li-Cor).

TDP-43 was detected with a polyclonal rabbit antibody at a 1:2,500 dilution (Proteintech 10782-2-AP). FUS was detected with a polyclonal rabbit antibody at a 1:2,500 dilution (Proteintech 11570-1-AP). hnRNPA1 was detected with a polyclonal rabbit antibody at a 1:5,000 dilution (Proteintech 11176-1-AP). DNAJB6 was detected with a monoclonal mouse antibody at a 1:2,500 dilution (Proteintech 66587-1-Ig). A goat anti-rabbit HRP conjugated antibody was used at a 1:50,000 dilution (Invitrogen G21234). A goat anti-mouse HRP conjugated antibody was used at a 1:10,000 dilution (Invitrogen 31430). All statistics were performed in GraphPad Prism Version 9.2.0.

Protein purification and in vitro LLPS experiments FUS-mEmerald was purified as described previously by Luo, Z. et al. (JCI Insight 5, 126769 (2019), incorporated herein by reference in its entirety). His-Sumo tagged DNAJB6 expression vector was transformed into E. coli BL21(DE3) (NEB) for protein expression and purification. DNAJB6 expressing cells were grown at 37° C. until an OD600 of 0.6 was reached. Expression was induced with 0.5 mM IPTG overnight at 16° C. Cell pellets were collected and subjected to high pressure lysis (Constant System) in lysis buffer (50 mMTris pH 7.5, 20 mM imidazole, 500 mM NaCl with 1× protease inhibitor cocktail). Lysate was centrifuged at 100,000×g and collected supernatant was applied to a 10 mL Ni-Advance (BioServ, UK) column. After washing, His-Sumo tagged DNAJB6 was eluted in 250 mM imidazole containing buffer and cleaved overnight with ULP-protease at 4° C. Cleaved DNAJB6 was diluted 5-fold before running through a cation exchange column. SP Sepharose chromatography was conducted in 50 mM HEPES, pH 7.5 with a salt gradient from 5 M−1 M NaCl. Fractions containing the protein were concentrated and subjected to size-exclusion on a Superdex-75 16/600 column in 50 mM HEPES and 100 mM NaCl, pH 7.5. Through all stages of purification, presence of DNAJB6 was monitored via SDS-PAGE.

DNAJB6 was labeled with Alexa Fluor™ 555 C2 Maleimide (Thermo Scientific) following the manufacturer's guidelines.

For LLPS experiments, concentrated, purified proteins (FUS, DNAJB6, and/or BSA) were diluted to 1.25 μM in a 50 mM NaCl solution unless otherwise stated. For imaging, condensates were maintained in PEG-silane coated Ibidi™ coverslides to avoid wetting. Imaging was conducted with Zeiss Axiovert 200M microscope with Improvision Openlab software using 100× magnification objective. Fluorescence recovery after photobleaching experiments were performed on a LSM780 (Carl Zeiss) using excitation from a 500 mW 488 nm OPSL. The resulting fluorescence was collected using an alpha Plan-Apochromat 63×/1.42 Oil immersion objective. The FRAP module on Zen Black (Carl Zeiss) was used to perform the experiment, with the FRAProfiler plugin (Fiji) from the Hardin lab used for analysis.

Mammalian Cell Lines and Cell Culture HEK293T cells used in this study were obtained from ATCC. Cells were maintained at 37° C. in a humidified atmosphere with 5% CO₂. HEK293T cells were grown in Dulbecco's Modified Eagle Medium (DMEM, Invitrogen) which was supplemented with 10% fetal bovine serum (Gibco) and penicillin-streptomycin (Invitrogen).

Mammalian Transfection 24 h prior to transfection, 293T cells were seeded at 40-60% confluency into 24-well plates coated for 30 min with a 0.1 mg/mL solution of poly-D-lysine (MP Biomedicals Inc.) and washed with PBS (Gibco) once prior to media and subsequent HEK293T cell addition. The next day, expression plasmid was incubated with Opti-MEM (Gibco) and Lipofectamine 2000 (Invitrogen) for 30 min at room temperature prior to addition to cells, per manufacturer protocol. 20 h after transfection, media was changed. Cells were harvested for protein extraction and western blotting 48 h after transfection.

Deep Mutation Screening Deep mutational scanning libraries were prepared in biological duplicates, wherein PCR mutagenesis, construction of bacterial libraries, and construction of yeast libraries were completed as independent replicates. The DNAJB6 yeast expression vector, pAG413GAL-DNAJB6 was miniprepped immediately prior to use. Variant versions of DNAJB6 were made by a single primer site-directed mutagenesis protocol. Oligos were designed to introduce a degenerate codon, NNK, at each amino acid position. Additionally, each oligo was designed to introduce 2-4 synonymous mutations at the codon immediately prior to the degenerate codon to increase sampling diversity. For each codon, an individual mutagenesis single primer PCR reaction was conducted in technical duplicates. The following PCR mix was used for mutagenesis: 5 μL 5× Q5 reaction buffer, 0.5 μL 10 mM dNTPs, 150 ng DNA, 1.25 μL 10 μM primer, and H2O to 25 μL. The following cycling conditions were used: 1. 98° C., 45 s, 2. 98° C., 15 s, 3. 60° C., 15 s, 4. 72° C., 260 s, 5. Return to step 2 29×, 6. 72° C., 240 s. After PCR, the unmodified backbone was digested with 1 μL DpnI at 37° C. for 1 h. After digestion, independent PCR replicates were pooled and sets corresponding to 14 contiguous amino acids were pooled together to enable data analysis using short read Illumina sequencing. 8 total sets were created encompassing 112 mutagenized amino acids. The combined sets were column purified with the Zymo DNA Clean & Concentrator Kit. Each biological replicate of each set was transformed into electrocompetent 10-beta E. Coli (New England Biolabs) in triplicate according to manufacturer instructions. Cells were plated following outgrowth and recovery on 15 cm LB agar plates containing ampicillin and colonies were allowed to form for 24 h at 30° C. An individual typical transformation yielded 3-10 million colonies for a total of approximately 9-30 million colonies for each biological replicate of each set. As a quality control measure, 20 colonies from each set were sequenced to ensure editing. All colonies were scraped off plates and plasmid libraries were purified by Midiprep (Zymo). Plasmid libraries were transformed into BY4741 containing the expression plasmid pAG416GAL-FUS. Each biological replicate of each set was transformed in 96 separate transformation reactions to ensure appropriate coverage and allowed 48 hours for outgrowth on SC-ura-his plates at 30° C. Yeast libraries were scraped into 10 mL sterilized PBS and frozen in 20% glycerol. For outgrowth, 600 μL of frozen yeast library was inoculated into 6 mL of SC-ura-his for 18 h in triplicate at 30° C. with rotation. After inoculation into galactose media, the remaining cells from the overnight cultures were spun down at 4,000 rpm for 5 minutes and the pellets were frozen at −20° C. For each independent culture outgrowth, 12 μL of saturated overnight culture was inoculated into 6 mL of SC-ura-his galactose for 48 h at 30° C. with rotation. Each tube was centrifuged at 4,000 rpm for 5 minutes and the supernatant was discarded. Pellets were resuspended in 300 μL of 200 mM LiOAc with 1% SDS and incubated for 15 minutes at 70° C. with shaking at 800 rpm. Afterwards, 900 μL 100% ethanol was added, tubes were vortexed, and centrifuged at 13,000 rpm for 10 minutes. Supernatant was discarded and pellets were allowed to air dry for 20 minutes under flame. Pellets were then resuspended in 200 μL TE and incubated at 42° C. for 20 minutes and then centrifuged at 13,000 rpm for 10 minutes. The supernatant containing DNA was collected and stored for further use. Each sample was then independently amplified and subsequently indexed for sequencing on a NextSeq 500/550 (Illumina). Depending on the set, amplification of the mutagenized region was done in 8 technical replicates that were pooled after amplification. The following mix was used for all PCR reactions: 5 μL 5× Q5 reaction buffer, 0.5 μL 10 mM dNTPs, 0.5 μL DNA, 0.125 μL 100 μM forward primer, 0.125 μL 100 μM reverse primer, 0.25 μL Q5 polymerase, and 18.5 μL H2O. Amplification was done for 24 cycles for non-induced libraries and 28 cycles for induced samples with the following conditions: 1. 98° C., 45 s, 2. 98° C., 15 s, 3. 58° C., 15 s, 4. 72° C., 30 s, 5. Return to step 2, either 24× or 28× 72° C., 240 s. After pooling, index sequences were attached using the same PCR mix and cycling conditions, but for 8 cycles of amplification. Amplicons were pooled according to set and amplicon length and gel purified to remove primers. Sequencing data were processed on Illumina Basespace according to default QC settings and downloaded as fastq files. Sequences were aligned using custom Python code. A raw activity score was calculated as:

$${Activity}_{i} = {{log}_{2} ( \frac{M u {t}_{i}}{W T} )}_{i n d u c e d} - {log}_{2} {( \frac{M u {t}_{i}}{W T} )}_{u n i n d u c e d}$$

where the subscript i denotes individual unique variants. Mutᵢ denotes the average number of counts of the particular mutant codon of interest, averaged over all codings, while WT denotes the number of counts of the wildtype nucleotide sequence. Raw activity scores were then normalized across sets by anchoring stop codon mutants to −1 and WT to 0 to eliminate set by set variation that may have arisen due to experimental fluctuation. The final normalized activity scores are presented in heatmap format for easy visualization. Plotted “wild type” values are derived from the recoded versions of the wild-type residue at a given position and thus do not always have a 0 value. Raw sequencing reads have been uploaded to the NCBI SRA under BioProject PRJNA769721 (SUB10503160).

Cas9 Knockout of DNAJB6 A Cas9 expressing HEK293T cell line was generated in a well of a 24-well dish by transfecting 300 ng of pB-CAGGS-Cas9-SV40-BPSV40 vector a long with 100 ng of a plasmid expressing the Piggybac transposase (System Biosciences, LLC) with Lipofectamine 2000 according to manufacturer protocols (Invitrogen). Media was changed 24 h after transfection before selecting with Noursethricin N-Acetyl Transferase (NAT) at 300 μg/mL 48 h after transfection. Cells were expanded and continuously selected with NAT for 2 weeks before being frozen down for further use.

A gRNA lentivirus compatible plasmid encoding two guide RNAs from the Brunello library (GCATATGAAGTGCTGTCGGA and GACTTCTTTGGGAATCGAAG (SEQ ID NOS: 2 and 3, respectively)) targeting DNAJB6 was created. Lentivirus was created from this plasmid by co-transfecting HEK293T cells with this plasmid alongside psPAX2 (Addgene #12260) and MD2.G (Addgene #12259). After transfection and a media change 24 h after transfection, media containing lentivirus was harvested 72 h later. Lentivirus containing media was added to Cas9 containing cells for 24 h before a media change. Beginning 48 h after the media change, cells were exposed to 2 weeks of alternating drug selections of NAT at 500 μg/mL and Blasticidin at 2 μg/mL every 48 h with regular splitting, to ensure both Cas9 and gRNA maintained good expression and were not silenced during the outgrowth process. After 2 weeks, single cells were sorted into 96 well plates using the Bigfoot Spectral Cell Sorter (Thermo). To gate on single cells, forward scatter and side scatter were used to isolate single cells and sort them into 100 μL of media. Single cells were allowed to expand for 2 weeks under alternating drug selection. DNA was harvested with QuickExtract (Lucigen) according to manufacturer protocols. PCR primers spanning individual cut sites in addition to primers spanning a potential deletion were used to amplify out the region of DNAJB6 subject to cutting. PCR products were sanger sequenced and TIDE was used to analyze sanger fragments to confirm disruption of DNAJB679. To confirm loss of DNAJB6, western blots were performed. The same process was repeated with non-targeting control guide RNAs (AAAAAGCTTCCGCCTGATGG and AAAACAGGACGATGTGCGGC (SEQ ID NOs: 4 and 5, respectively)).

RNA-seq HEK293T cells were transfected with 50 ng of expression plasmid and grown for 72 hours in a 24 well dish after transfection. Cells were harvested in TRIzol and stored at −80° C. RNA was harvested from cells with the Direct-zol miniprep kit (Zymo). Harvested RNA was prepared for sequencing with the NEBNextR Ultra™ II RNA Library Prep Kit for Illumina (NEB). Two biological replicates were performed for each condition. Each individual replicate was amplified with a unique combination of indexing primers after the adaptor ligation step to uniquely identify it. Pooled libraries were combined and sequenced with a 75 cycles NextSeq 500/550 High Output Kit on a NextSeq 550 machine (Illumina). Each replicate was allocated ˜30 million reads. Reads were aligned to the hg19 genome using HISAT2 to obtain counts. Differential expression was calculated using limma. Raw sequencing reads have been uploaded to the NCBI SRA under BioProject PRJNA769721 (SUB10426285).

Human ORFeome Library construction The pooled hORFeome V8.1 library was inserted into the pAG413GAL-ccdb-6Stop vector with Gateway LR II Clonase Enzyme mix (Invitrogen) at a ratio of 150 ng:50 ng. The reaction was incubated overnight at 25° C. Expression plasmids were electroporated into electrocompetent 10-beta E. Coli (NEB) and ˜200,000 colonies were harvested and Miniprepped. The expression plasmid library was transformed into BY4742 and selected in SC-his glucose plates for 48 hours. Individual colonies were picked and arrayed into 96 well plates and saved. To identify the ORF present in each well, each plate was process individually. A total of 20 pools per plate were made, consisting of 12 column pools and 8 row pools. DNA from each of these pools was then obtained using a LiOAc-based extraction. ORFs within each pool were amplified for 30 cycles with general primers binding to the galactose promoter and cyc terminator and subsequently column purified. 250 ng of the purified PCR product was processed with the NEBNext Ultra II FS DNA Library Prep Kit for Illumina (NEB) according to manufacturer protocols. After adaptor ligation and prior to indexing, a forward primer placed 60 bp upstream from the ATG start codon was used in combination with an adaptor reverse primer and amplified for 13 cycles to selectively enrich for the human ORF containing fragment in preparation for sequencing. Each individual pool was then uniquely indexed and sequenced with a 150 cycles NextSeq 500/550 High Output Kit on a NextSeq 500/550 machine (Illumina). After sequencing, the identity of each well was determined by its presence in a specific combination of row and column wells.

### EXAMPLE 1

### Development of a Multiplexed Screening Strategy to Identify Rescuers of Proteotoxicity

To simultaneously study dozens of disease models at a time, isogenic yeast strains that each contain a unique DNA-barcode inserted into the same neutral genomic locus were used. Into each of these barcoded strains, a construct encoding a proteotoxic species, such as an NDD-associated protein (e.g., TDP-43 or FUS) is delivered. Growth of these strains in media that induces the expression of the toxic disease-associated proteins causes a reduction in cell growth. By associating the different models with particular DNA-barcodes, they can be combined into a single mixed pool and the growth of each member can be tracked by measuring its barcode abundance in the pool using next-generation sequencing.

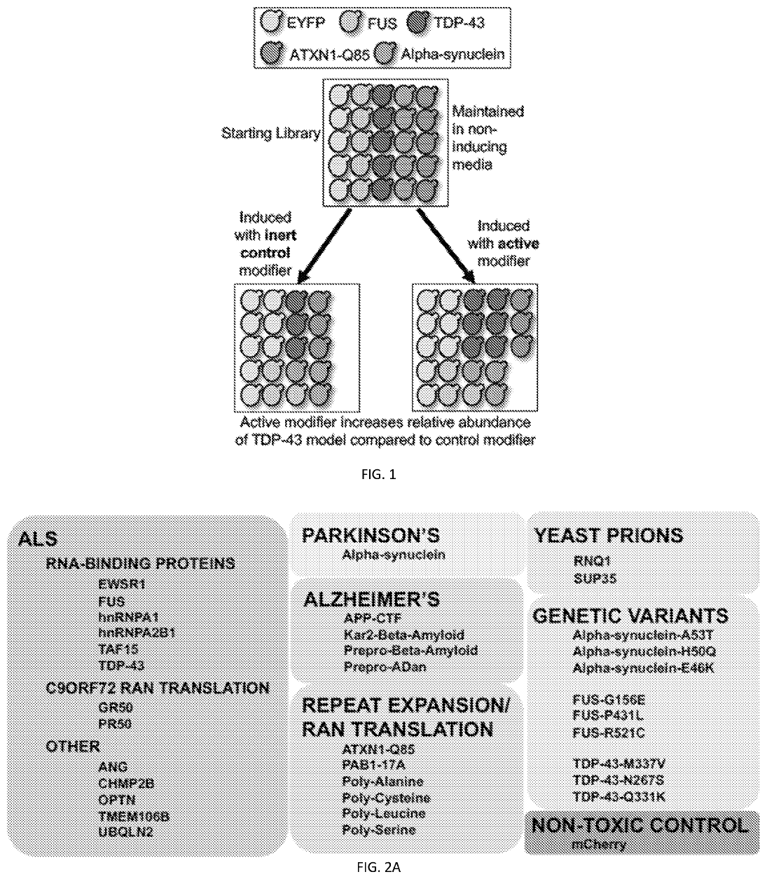

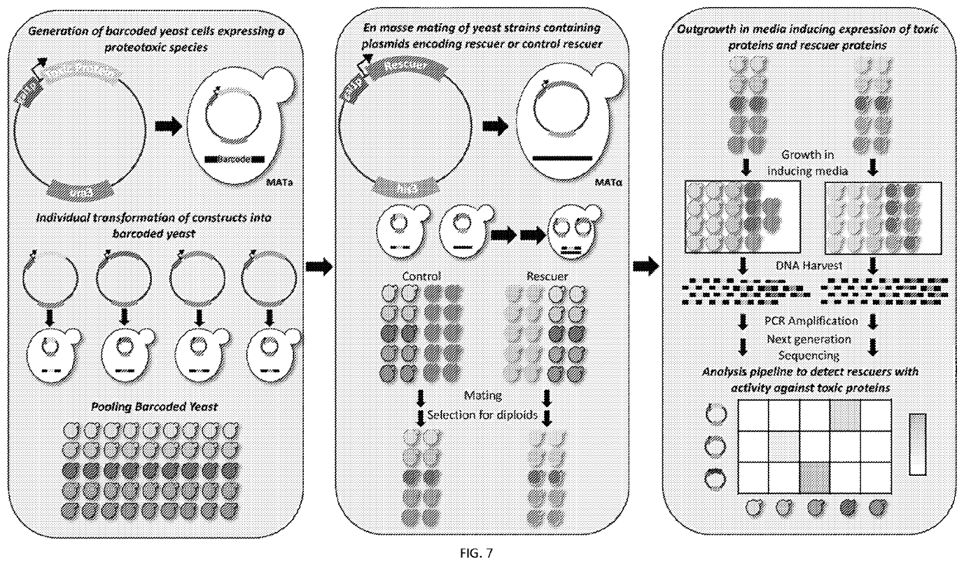

To identify novel regulators of neurodegeneration, the pool of disease models was probed against a library of genetic modifiers (FIGS. 1 and 7). In cases where a genetic modifier suppressed the toxicity of a particular NDD-associated protein, a marked increase in the abundance of the model's barcode was observed as compared to the control condition. Finally, taking advantage of the scalability afforded by the use of DNA-barcoding, each model within the pool was placed into several different DNA-barcoded strains (e.g., redundant barcoding). This decreased assay noise by allowing use of the collective behaviors of all uniquely barcoded strains containing the same model to derive conclusions and enables confident identification of significant interactions between the disease models and genetic modifiers.

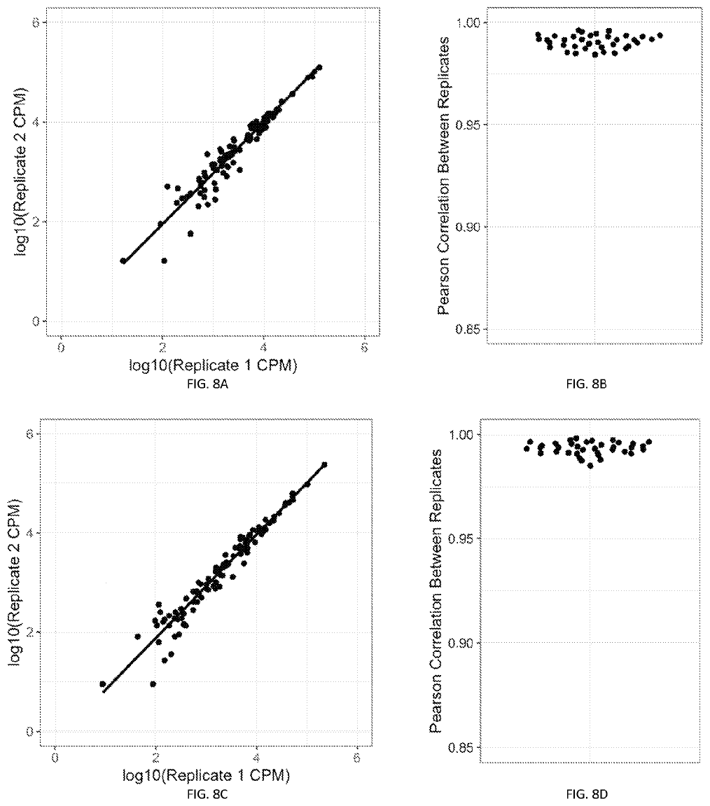

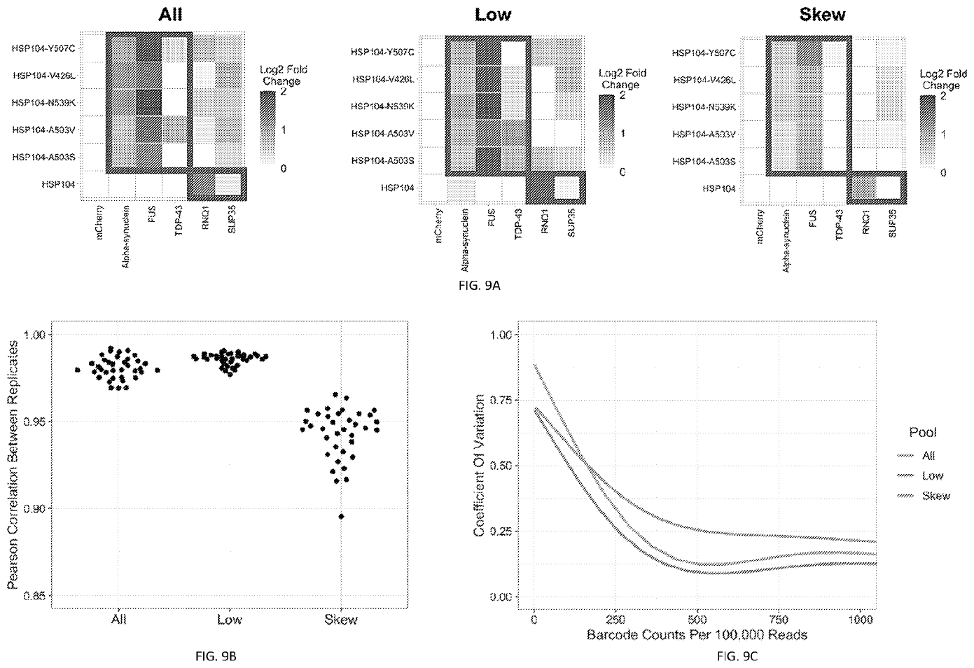

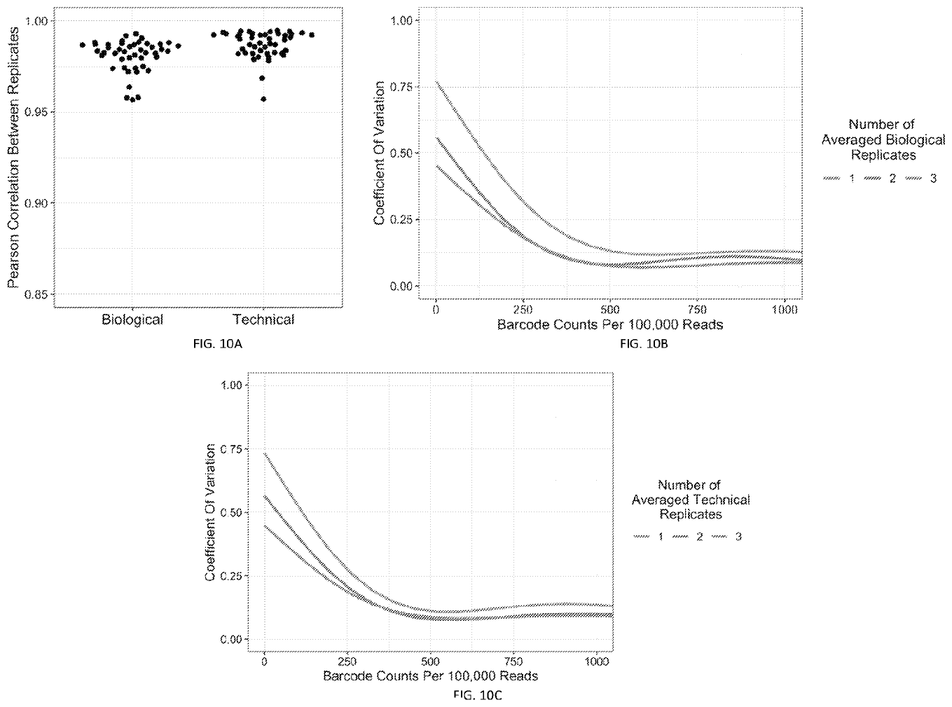

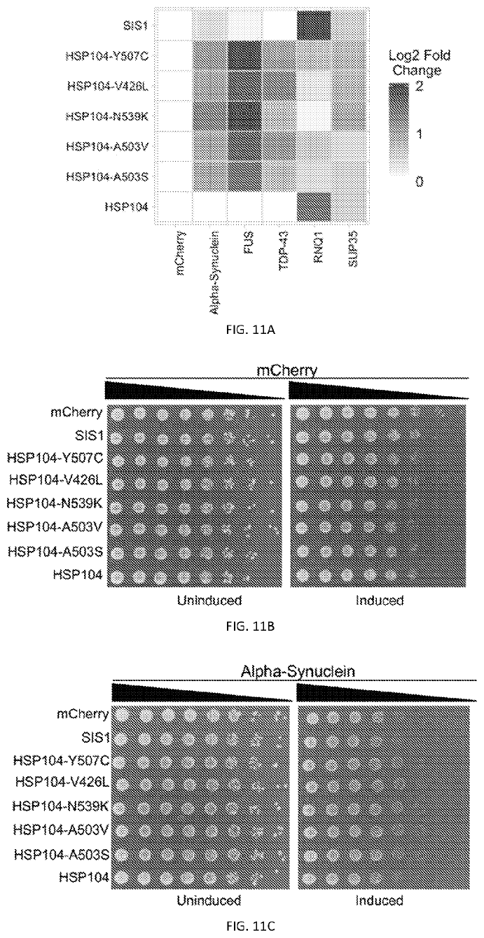

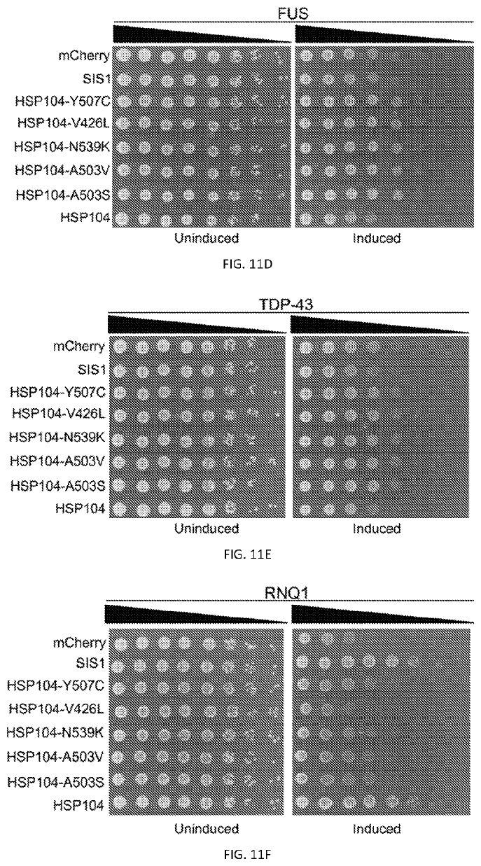

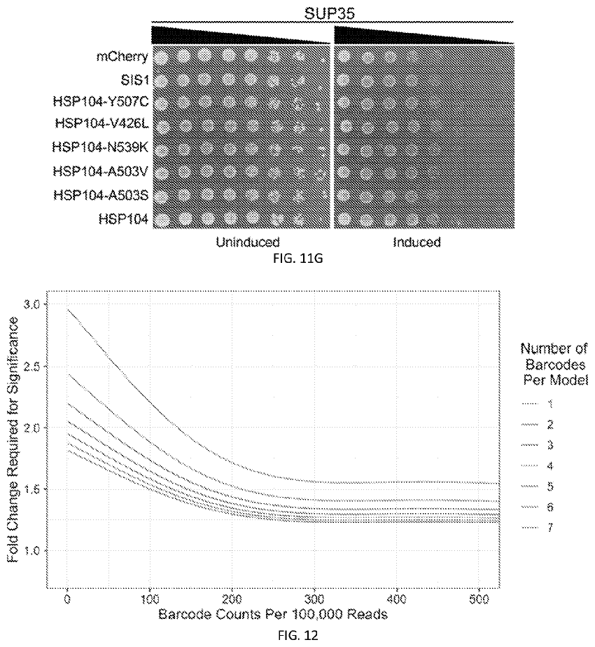

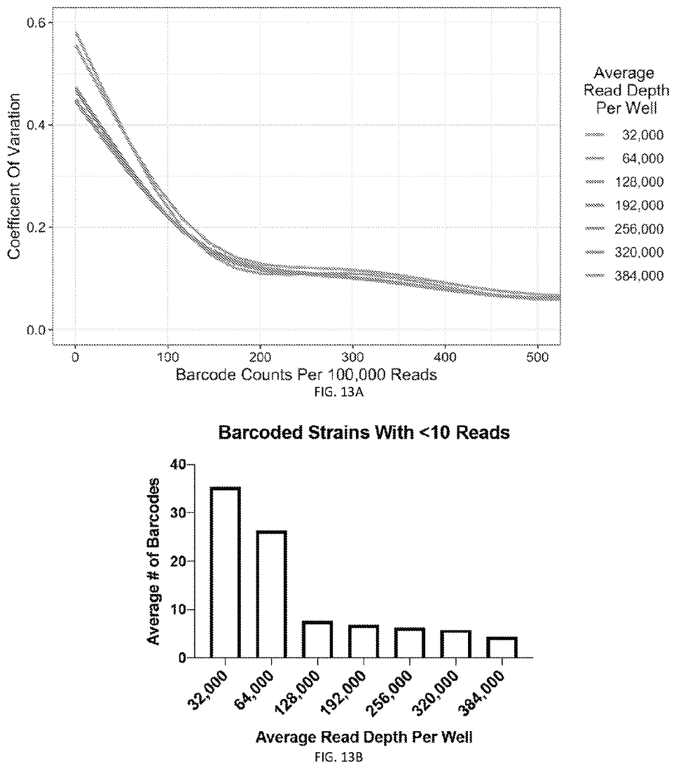

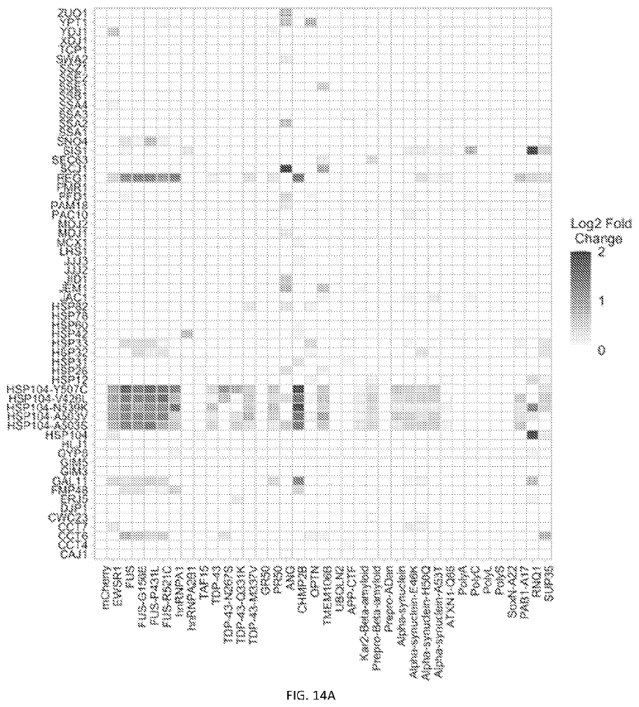

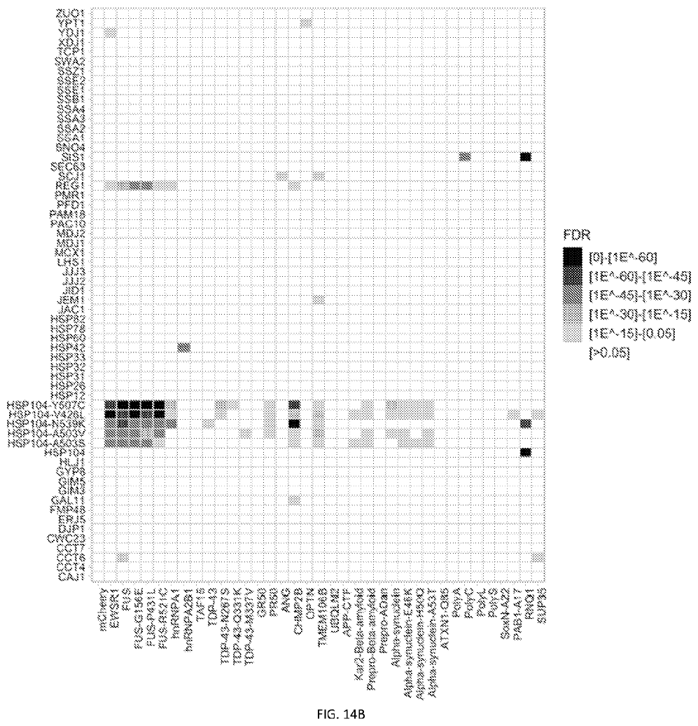

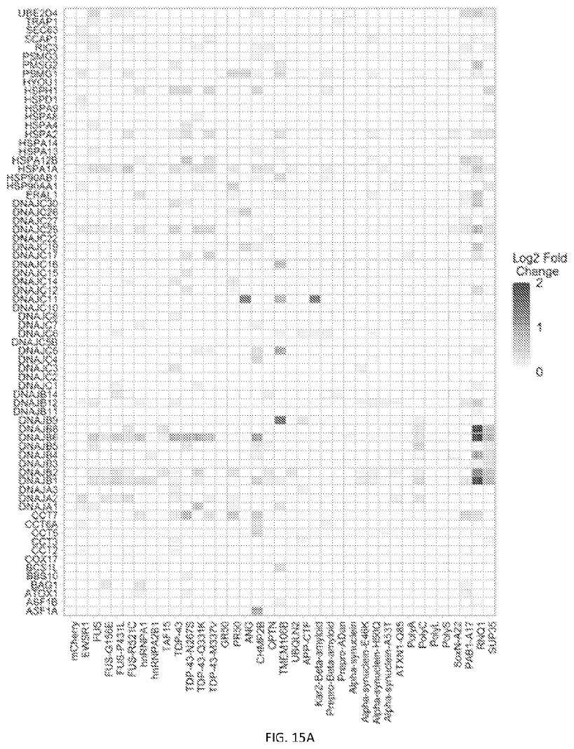

To develop the approach, reproducibility throughout the screening process was analyzed, optimal strategies for pooling DNA-barcoded strains were identified, the appropriate number of experimental replicates was determined, and the amount of redundant barcoding required to detect interactions at high sensitivity was modeled (FIGS. 7-13). With these optimized parameters, a large collection of 35 models of neurodegeneration and protein misfolding were then assembled based on previous publications (FIG. 2A). For a subset of the models, patient-derived mutant variants that are associated with an increased likelihood of disease were also included in order to determine how these mutations might influence the observed rescue (FIG. 2A). Using the final pool of 302 DNA-barcoded strains, the amount of sequencing depth needed per condition tested was determined before proceeding to perform the screens (FIG. 13).

### EXAMPLE 2

### Functional Characterization of Chaperone Interactions with Neurodegenerative Disease Models

Having established the screening pipeline, a targeted library of 132 molecular chaperones, 62 of which were from yeast and 70 from humans, were probed. Molecular chaperones have been implicated in the refolding, turnover, and mitigation of the toxicity of aggregation-prone proteins implicated in neurodegeneration. However, a comprehensive map of the functional interactions between chaperones and their disease associated clients remains elusive, limiting our ability to identify broadly-active members of this class of proteins. Overall, this screen represented 5,850 genetic interactions between the various models and molecular chaperones.

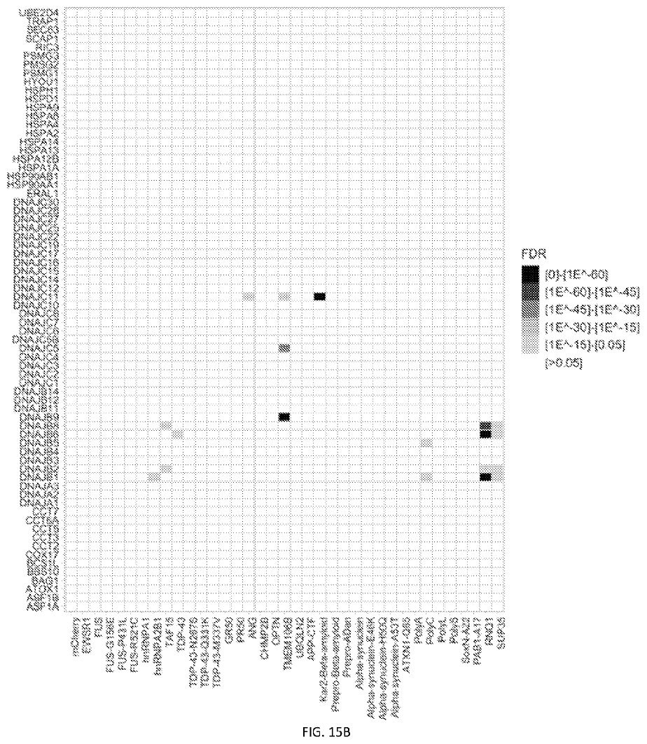

Within the yeast chaperone set, 112 strong interactions were observed that resulted in a greater than 0.5 log2 fold increase in barcode abundance for a number of models in the pool (FIGS. 2B and 14A). Notably, potentiated HSP104 chaperones were broadly active with the ability to rescue the proteotoxicity of a number of models including two beta-amyloid models, hnRNPA1, and the dipeptide repeat PR50 associated with c9orf72 RAN-translation, in addition to their previously reported rescue of TDP-43, FUS, and alpha-synuclein models. Outside of strong interactions, such as those observed with the HSP104 variants, 74 interactions with mild-to-moderate positive log2 fold changes between 0.25 and 0.5 were also observed. In order to prioritize the 186 interactions for further follow up, an analysis pipeline was developed to call interactions with statistically significant enrichment (FIGS. 2C and 14B). This pipeline used the control wells with inert rescuers to develop an expectation for the abundance of each barcode in the pool, determined which barcodes significantly increased in abundance in test wells, and combined information from barcodes associated with the same model to identify the most potent and significant hits for further interrogation (see Materials and Methods). Upon applying the data analysis pipeline, 100 interactions were called as significant. Among these significant interactions, specific interactions were identified between the ALS/FTD-associated RNA-binding proteins, EWSR1, FUS, and hnRNAP2B1 and the type I HSP40 chaperone YDJ1, the chaperonin containing TCP-1 (CCT) subunit CCT6, and the small heat shock protein HSP42, respectively. An interaction was also observed between the Golgi-maintenance protein and autophagosomal receptor OPTN and a Rab family GTPase involved with ER-to-Golgi transport that localizes to pre-autophagosomal structures, YPT1 (FIG. 2D).

The mammalian chaperone set contained 131 interactions between proteotoxic models and chaperones that resulted in mild, moderate, or strong log2 fold changes in barcode abundance (FIGS. 2E and 15). However, 86 of the 131 interactions led to mild-to-moderate changes in barcode abundance, possibly reflecting the suboptimal function of mammalian chaperones within yeast cells (FIGS. 2E and 15A). Nevertheless, 20 significant interactions were identified, including rescue mediated by the primarily mitochondria-localized type III HSP40chaperone, DNAJC11, and two membrane associated models, TMEM106B and Kar2-beta-amyloid (FIGS. 2F, 2G and 15B). These results are of interest as DNAJC11 mutant mice show prominent neuronal pathology with vacuolization of the endoplasmic reticulum and disruption to mitochondrial membranes. Furthermore, both TMEM106b and beta-amyloid cell-based models have been associated with mitochondrial stress and dysfunction. These findings suggest that the observed rescue with DNAJC11 may be related to its critical role in membrane integrity and buffering the cell against neurotoxic insults such as those from beta-amyloid. Given the breadth of the screening it enabled the detection of a general trend of interaction between multiple human type II HSP40 chaperones (DNAJB1, DNAJB2, DNAJB4, DNAJB6, DNAJB8) and two yeast prions, RNQ1 and SUP35 within the library. These findings indicate that the misfolded intermediates produced by these two yeast prions may have similar properties, which is in agreement with data showing the ability of these two yeast prions to cross-seed each other's aggregation. Furthermore, the fact that SIS1, the yeast orthologue of these human DNAJB proteins, shows prominent activity against RNQ1 and SUP35, suggested that this class of misfolded prion species may be evolutionarily conserved substrates for this family of chaperones.

### EXAMPLE 3

### Secondary Validation of Multiplexed Screening Results

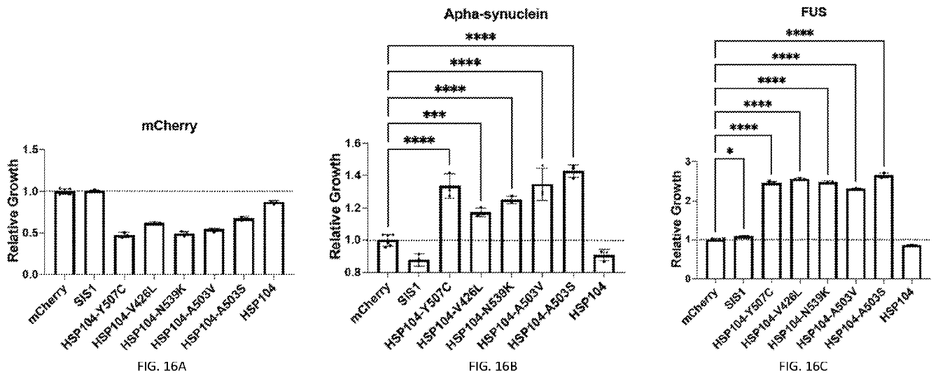

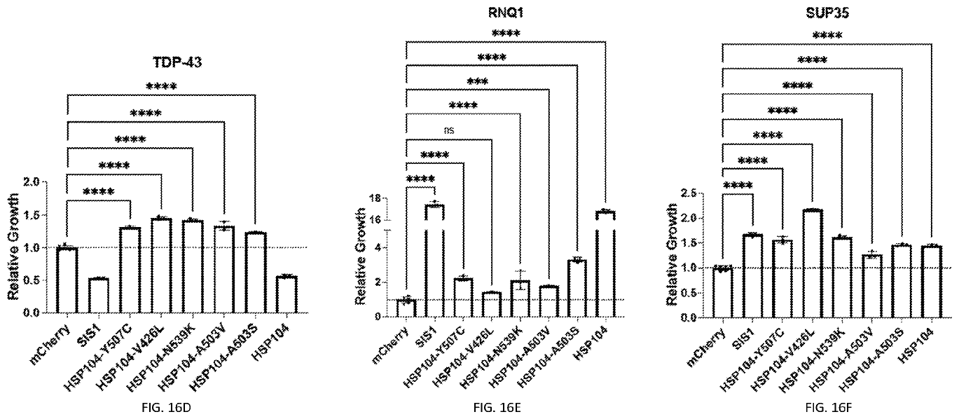

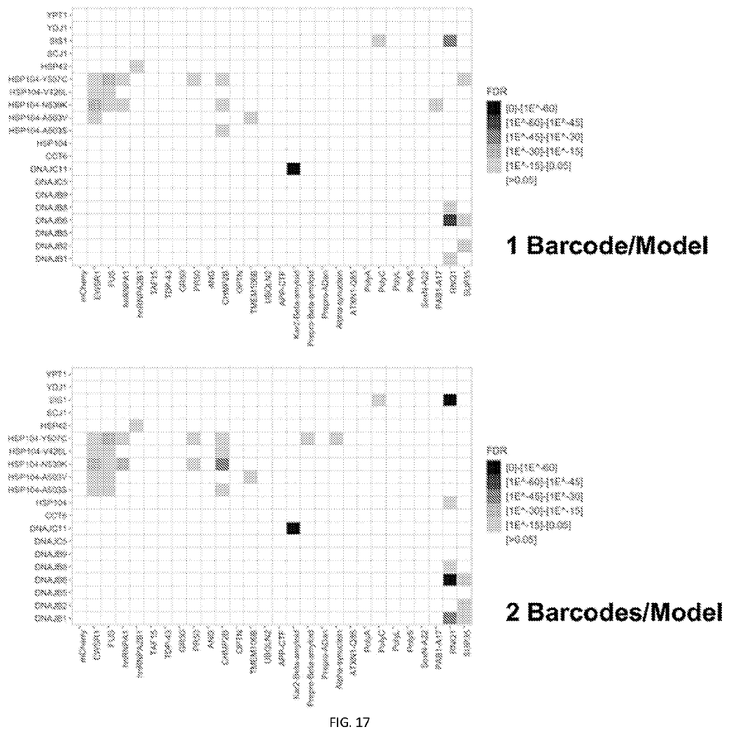

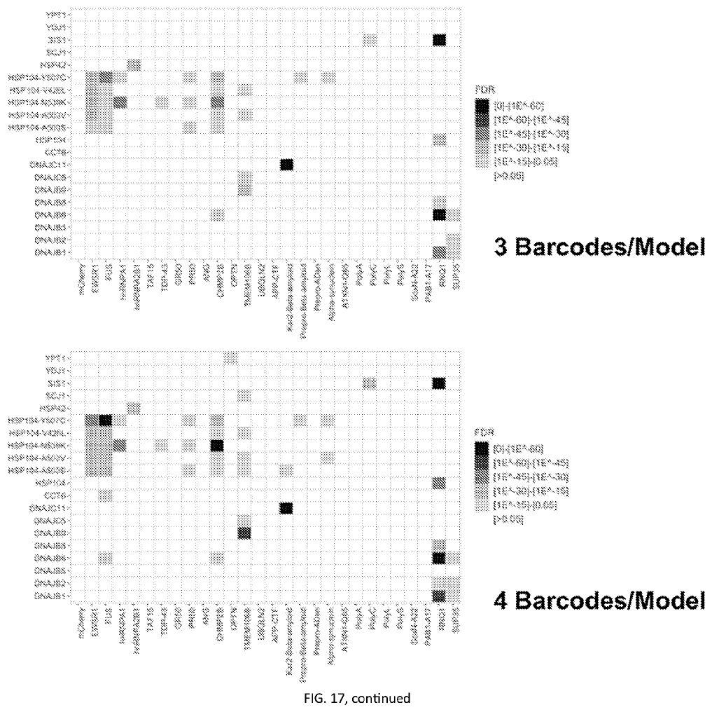

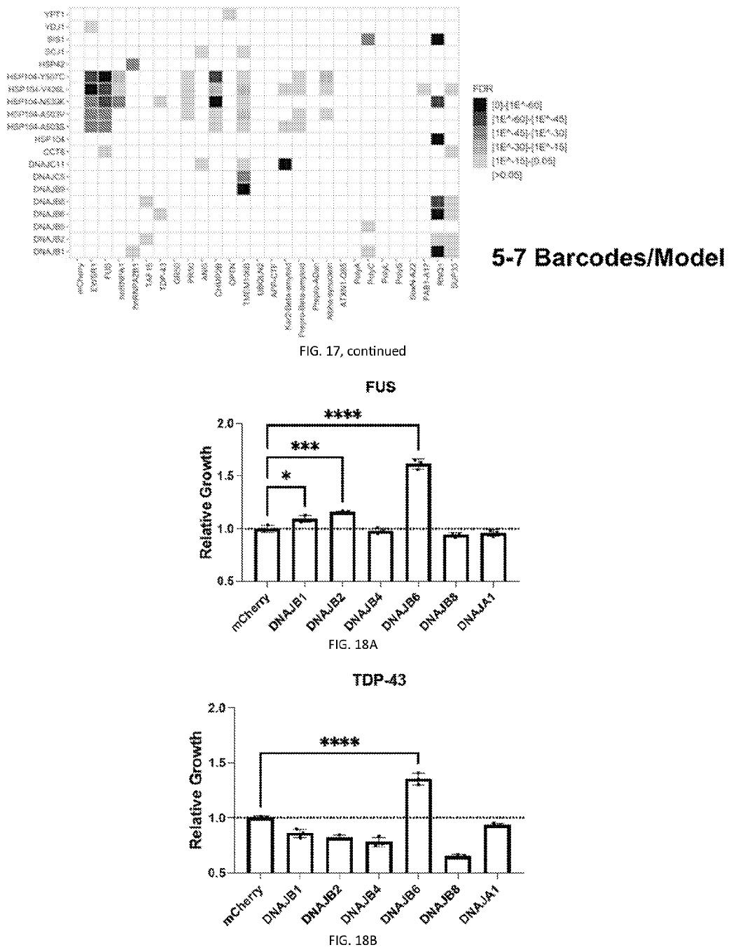

To validate the interactions identified in the screen, a testing paradigm similar to how the screen was conducted. For this purpose, a previously reported passaging-based growth assay (See, Breslow, D. K. et al. Nat Methods 5, 711-718, (2008), incorporated herein by reference) was used to first verify against a set of known interactions (FIG. 16). The assay was then used to individually validate two sets of hits, those judged as statistically significant by our analytical pipeline and those with positive log2 fold changes that did not reach our significance threshold but represent “suspected interactions”. Hits identified as statistically significant with an FDR adjusted p-value of <0.05 were validated at a high rate, with 116/120 (96.7%) of these interactions reproducing upon individual testing. Furthermore, highlighting the power of the redundant barcoding strategy, when the number of barcodes analyzed for each model was progressively reduced from 5-7 redundant barcodes per model to 1 per model, a striking decrease in the number of hits captured was seen with each barcode removed (FIG. 17). These results reinforce the dramatic improvement in data quality afforded by transforming each model into multiple DNA-barcoded strains and analyzing their collective behavior to empower the hit calling algorithm. A number of suspected interactions with positive log2 fold changes that did not meet statistical significance were next verified, with 59/95 (62.1%) of these suspected interactions showing rescue upon individual testing. This indicated that the hit calling algorithm prioritized interactions that are likely to validate over log2 fold changes alone, although some true hits may be missed as these likely would not survive the adjustments for multiple hypothesis testing. To estimate the sensitivity and specificity of the statistical pipeline, it was assumed that the 175 validated interactions (116 significant and 59 suspected) represented most of the hits in the matrix of 5,850 interactions. Holding this to be the case, it suggested that the screening platform had an estimated sensitivity and specificity of ˜66% and ˜99%, respectively, on par or better than previously reported one-model-at-a-time screening approaches.

### EXAMPLE 4

### DNAJB6 is a Rescuer of Multiple RNA-Binding Proteins Implicated in ALS/FTD

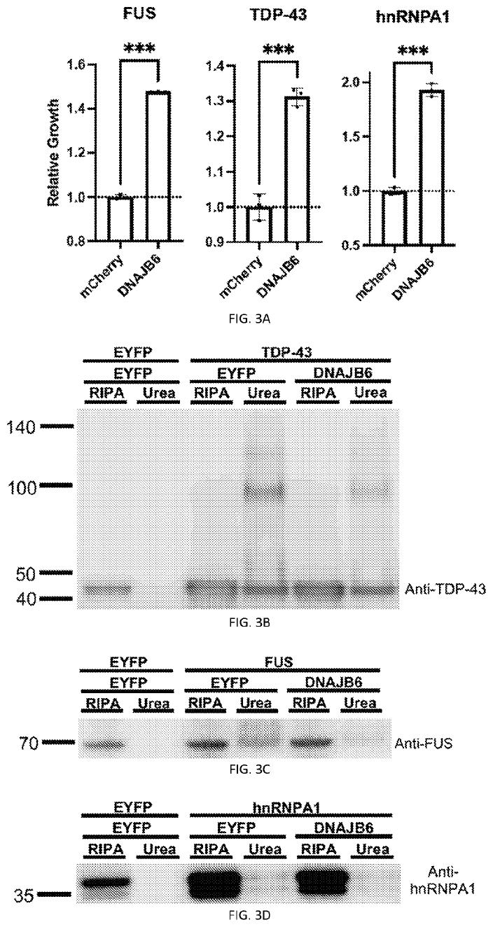

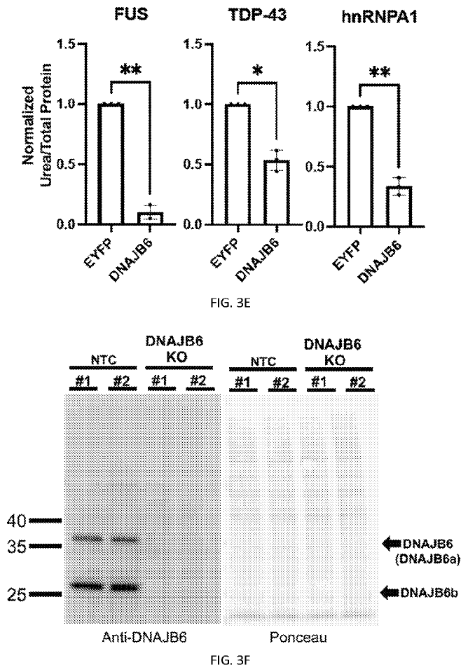

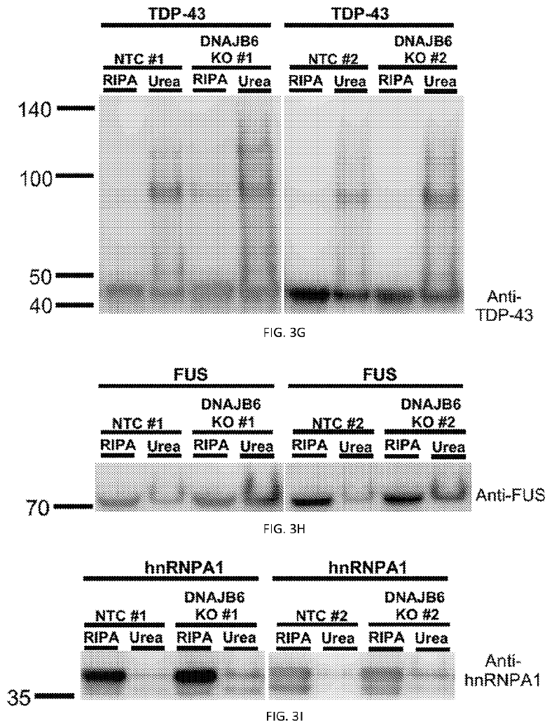

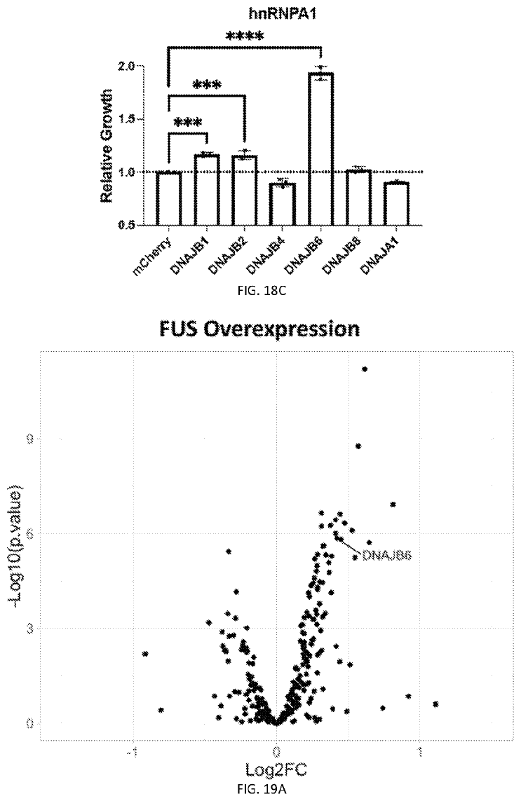

During screening and subsequent validation, a human chaperone, DNAJB6, was identified that rescued the toxicity of cells expressing ALS/FTD-associated aggregation-prone RNA-binding proteins FUS, TDP-43, or hnRNPA1 (FIG. 3A). DNAJB6 is a type II HSP40 co-chaperone expressed in a number of tissues, including ubiquitously throughout the brain and spinal cord. However, DNAJB6 expression is decreased in the brain with aging and is downregulated upon differentiation of induced pluripotent stem cells into neurons, suggesting that depleted DNAJB6 levels may sensitize neurons to misfolded protein stress. HSP40 co-chaperones comprise a large class of approximately 50 proteins in humans with a broad-spectrum of reported functions, but primarily function by binding to misfolded proteins, trafficking misfolded proteins to HSP70 chaperones, and regulating protein-protein interactions. While other type II HSP40s were able to rescue FUS, TDP-43, or hnRNPA1 yeast models, DNAJB6 was unique in the magnitude of its effect and the ability to rescue all three models (FIG. 18).

In order to determine if the interaction between DNAJB6 and the RNA-binding proteins FUS, TDP-43, and hnRNPA1 are relevant within mammalian cell contexts, an in vivo protein aggregation assay was employed. In this assay, when FUS, TDP-43, or hnRNPA1 are overexpressed within human embryonic kidney 293T (HEK293T) cells, they form SDS-insoluble (RIPA buffer insoluble), urea-soluble species (FIGS. 3B-3D). As compared to control cells co-transfected with enhanced yellow fluorescent protein (EYFP), cells co-transfected with DNAJB6 showed a reduction in the amount of SDS-insoluble species for FUS, TDP-43, and hnRNPA1 (FIGS. 3B-3E). This demonstrated that DNAJB6 is capable of reducing the formation of insoluble species for multiple RNA-binding proteins within mammalian cell contexts.

### EXAMPLE 5

### Endogenous DNAJB6 Participates in the Response to Accumulation of Insoluble FUS, TDP-43, and hnRNPA1

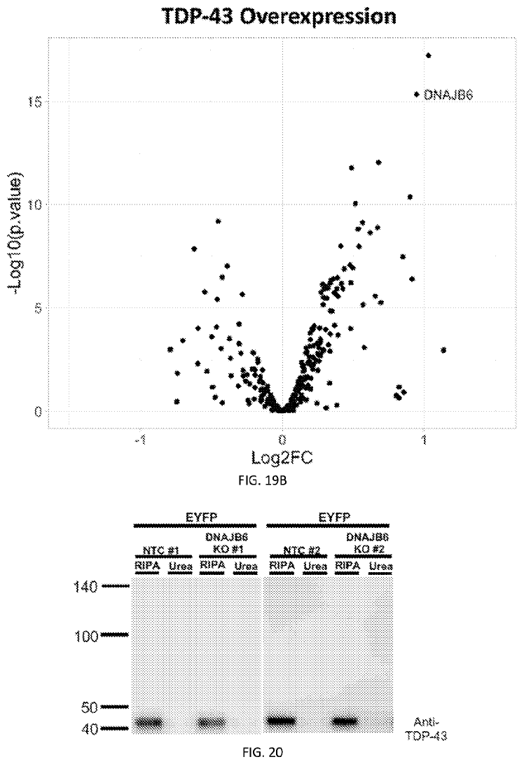

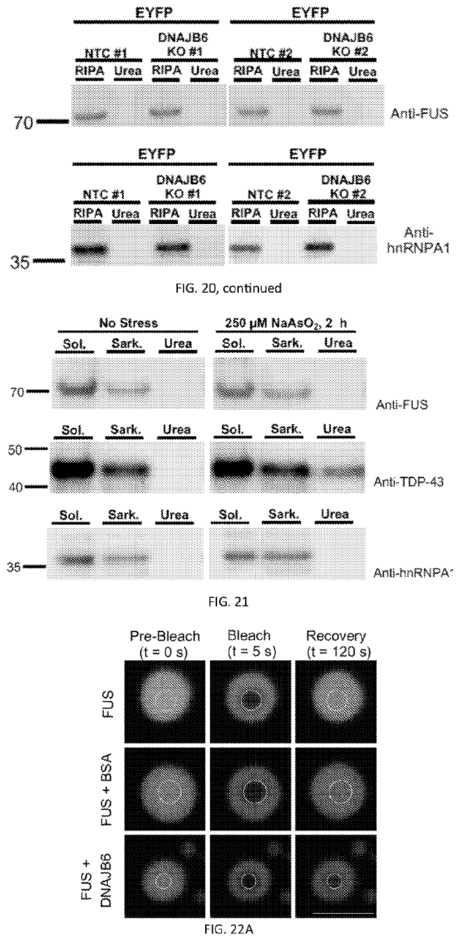

To further investigate the role DNAJB6 plays in the response to increasing cellular concentration of ALS/FTD-associated RNA-binding proteins, HEK293T cells were transfected with FUS or TDP-43 overexpression constructs and performed unbiased RNA-sequencing. In both cases, DNAJB6 was one of the most strongly and significantly upregulated chaperones among the ˜270 molecular chaperones expressed in HEK293T cells (FIG. 19). To determine what role endogenous levels of DNAJB6 play in regulating FUS, TDP-43 and hnRNPA1 levels, Cas9 was used to generate multiple independent clones in which DNAJB6 was knocked out (FIG. 3F). In DNAJB6 knockout lines, the formation of SDS-insoluble FUS, TDP-43, or hnRNPA1 species was not observed when they were expressed at endogenous levels (FIG. 20). However, upon overexpression of FUS, TDP-43, or hnRNPA1, greater amounts of SDS-insoluble species within DNAJB6 knockout lines were observed as compared to our non-targeting gRNA control (NTC) lines (FIGS. 3G-3J). These data suggested that DNAJB6 is part of programmed cellular response to rising levels of multiple aggregation-prone RNA-binding proteins and that DNAJB6 can help regulate their solubility when present at physiologic levels.

### EXAMPLE 6

### DNAJB6 Can Regulate Protein Phase Changes in Response to Cellular Stress

Stress granules (SG) are transient liquid-liquid phase separated (LLPS) RNA-protein granules that are induced by cellular stress. Many of the RNA-binding proteins associated with ALS/FTD share a common feature of being recruited to these condensates via their intrinsically disordered domains (IDRs). Over time, if these condensates are not resolved, the proteins within them may aggregate. Stress granule induction was used to determine whether native levels of DNAJB6 play a role in regulating the solubility of endogenously expressed FUS, TDP-43, or hnRNPA1 and their misfolding. To increase the sensitivity for detecting changes in protein solubility, a 3-part protein extraction employing a buffer containing sarkosyl instead of SDS was utilized as sarkosyl is a gentler detergent. While sodium arsenite stress did not change the solubility of natively expressed FUS or hnRNPA1 in HEK293T cells, it did alter the solubility of endogenously expressed TDP-43, resulting in the formation of sarkosyl-insoluble, urea-soluble TDP-43 species (FIG. 21). When this experiment was performed in DNAJB6 knockout and control lines, the relative proportion of TDP-43 in the urea-soluble, sarkosyl-insoluble fraction was increased in the DNAJB6 knockout lines compared to the NTC lines (FIGS. 4A-4B). Furthermore, HEK293T cells overexpressing DNAJB6 resolved the formation of these urea-soluble, sarkosyl-insoluble TDP-43 species more robustly than cells overexpressing EYFP (FIGS. 4C-4D). These results showed that at native levels of expression DNAJB6 plays a key role in maintaining the solubility of TDP-43 during times of stress.

### EXAMPLE 7

### DNAJB6 Undergoes Phase Separation and Can Prevent FUS Aggregation In Vitro

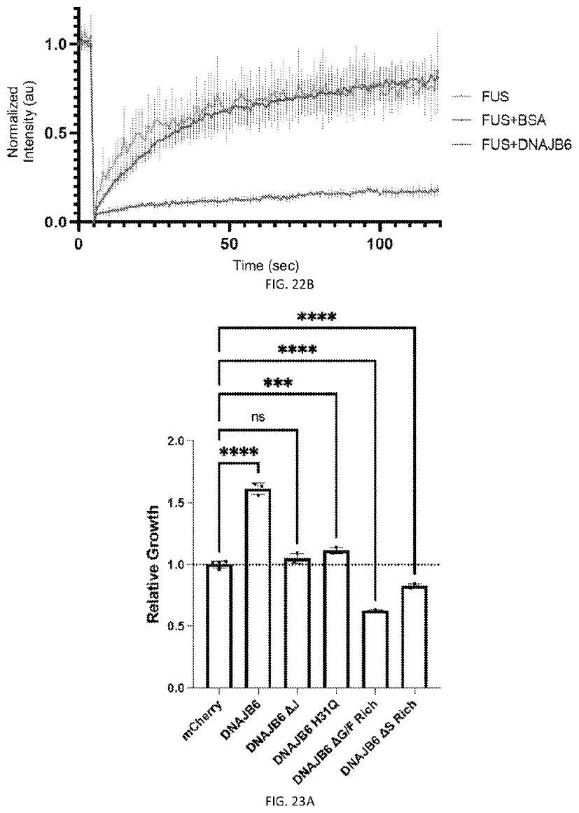

While physiologic condensates can contain hundreds of proteins and RNAs, model systems composed of a single protein such as FUS, with an intrinsic ability to form condensates in vitro, have been instrumental in elucidating the complex biophysics underlying these unique species, including how condensates can “mature” and aggregate over time. To test if DNAJB6 might carry out its function by partitioning into condensates containing its client proteins, DNAJB6 was purified and its ability to undergo LLPS in vitro was assessed. DNAJB6 underwent LLPS at near-physiologic salt concentrations (FIG. 4E). When mixed 1:1 with purified mEmerald tagged FUS, DNAJB6 readily co-partitioned with FUS condensates (FIG. 4F). During these mixing studies, DNAJB6 decreased the size of FUS condensates, as compared to the bovine serum albumin (BSA) control (FIGS. 4G-4H). These smaller FUS condensates, which form in the presence of DNAJB6, could represent species that were less liquid-like and as such were less likely to fuse. To confirm a change in the biophysical properties of the intermixed FUS and DNAJB6 condensates, fluorescence recovery after photobleaching (FRAP) studies were performed (FIG. 22A). As compared to FUS alone or FUS in the presence of control protein BSA, DNAJB6 decreased the recovery of co-incubated FUS within the condensate, in agreement with FUS being in a more gel-like state (FIG. 22B). Over longer periods of time, condensates containing FUS alone have a propensity to undergo a transition from liquid or gel-like condensates to solid aggregates. Over 48 hours of incubating FUS at physiological salt concentrations, the appearance of multiple, large disordered aggregates was observed (FIG. 4I). However, no aggregates were formed over 48 hours when FUS was incubated in the presence of equimolar amounts of DNAJB6 (FIG. 4I), suggesting that DNAJB6 can rapidly convert FUS into a stable gel-like state for extended periods of time and can block FUS aggregation. These results are in line with the cellular findings, which observed that DNAJB6 blocked the proteotoxicity and aggregation of multiple proteins with the propensity to undergo LLPS.

### EXAMPLE 8

### Characterization of DNAJB6 via Domain Deletions and Deep Mutational Scanning

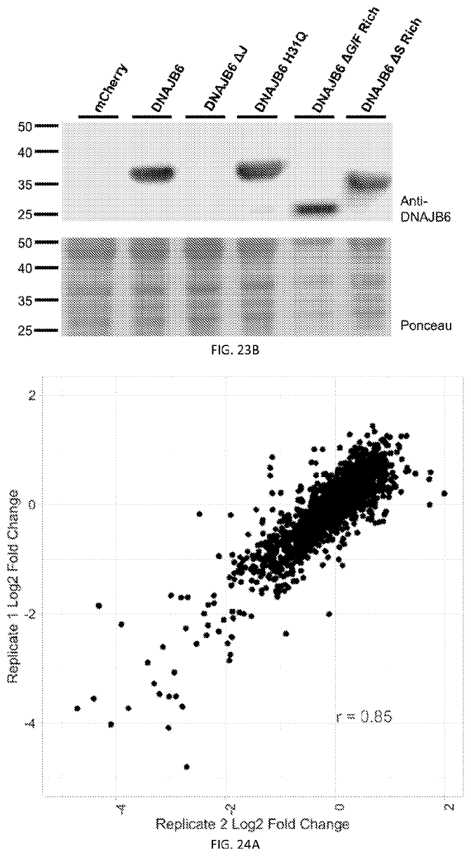

To decipher the regions within DNAJB6 required for its activity, versions of DNAJB6were constructed lacking its J domain which is necessary for activation of HSP70 partners, its glycine/phenylalanine rich region which contributes to its ability to phase separate and partition with client proteins, or its serine rich domain that is implicated in client recognition and binding. Disruption of any domain of DNAJB6 prevented it from rescuing the toxic effects of FUS on cell growth, consistent with results seen for a related HSP40 chaperone, DNAJB1, and its interaction with FUS (FIG. 23A). All constructs were expressed in yeast except for the variant with the J domain deletion, prompting testing of a mutant within the conserved HPD motif of the J domain (H31Q) that blocks its ability to stimulate the ATPase activity of HSP70 family members. The H31Q mutation also rendered DNAJB6 non-functional for rescuing FUS-mediated toxicity. These results suggested that all domains within DNAJB6 facilitate its activity and that cooperating with a HSP70 partner may be important for its full function in vivo (FIGS. 23A-23B).

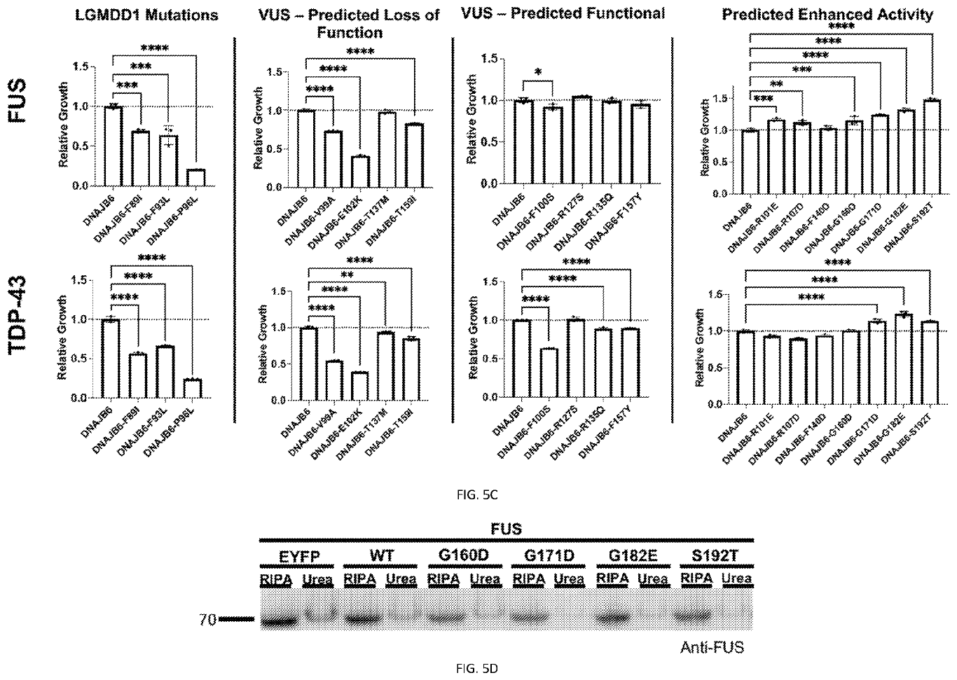

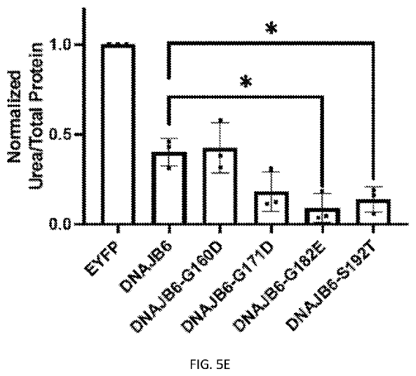

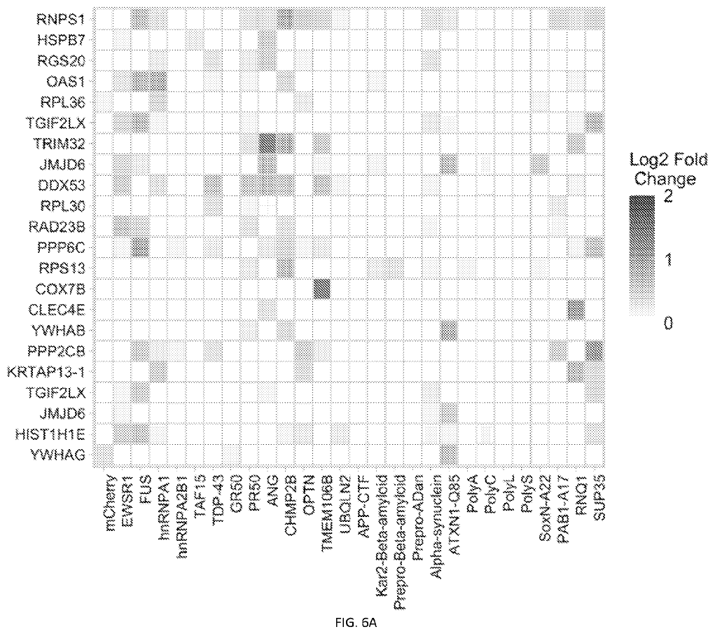

During the deletion analysis, it was observed that loss of the glycine/phenylalanine (G/F) rich domain or the serine (S) rich domain enhanced the toxicity of the FUS model (FIG. 23B). These regions have been implicated in substrate recognition and contain LGMDD1 patient mutations along with multiple variants of uncertain significance within the Clin Var database. To decipher the function of these critical residues at increased resolution, a deep mutational scan (DMS) was conducted across the 112 amino acids of the G/F and S-rich regions. Deep mutational scanning libraries were constructed by performing comprehensive mutagenesis of each codon, yielding versions of DNAJB6 with all 20 amino acids or a stop codon at each interrogated position (FIG. 5A). The library of variants was then tested for their ability to rescue the proteotoxicity induced upon FUS overexpression, enabling generation of a comprehensive fitness landscape of all single amino acid mutations in the G/F and S rich region on DNAJB6 activity (FIG. 5B). Mutagenesis libraries were constructed and screened in biological duplicates, with strong correlation between replicates observed (see Materials and Methods, FIG. 24). Notably, regions rich in patient mutations causing LGMDD1 (amino acids 89-100) frequently resulted in a strong reduction in activity, with subsequent validation of these mutations confirming a loss of activity compared to wild type (WT) DNAJB6 against the FUS model (FIGS. 5B-5C). Similar results were obtained when these mutants were tested against the TDP-43 model, suggesting the existence of a common mechanism of interaction between DNAJB6 and its aggregation-prone clients (FIG. 5C). Numerous variants of uncertain significance (VUS) in DNAJB6 present in ClinVar were also captured within the data. Follow-up studies individually examining these mutants revealed a subset which were defective in their ability to rescue FUS and TDP-43 expressing models, in agreement with the DMS results (FIG. 5C). Within the DNAJB6 mutational landscape, numerous variants that were predicted to enhance its activity on FUS were also observed. Outside of the conservative S192T mutation which showed one of the strongest effects on rescue, a general trend was seen where mutation to an acidic residue in stretches between 138-171 and 182-189 appeared to enhance activity compared to WT DNAJB6 (FIG. 5B). In agreement with this observation, the single acidic amino acid within these stretches, D158, facilitated activity as mutation of D158 to almost any other amino acid other than glutamic acid reduced the activity of the protein (FIG. 5B). A series of mutations predicted by DMS to enhance the activity of DNAJB6 were validated against both the FUS and TDP-43 models, finding 3 mutations that showed clear gains in activity as compared to the wild-type protein (FIG. 5C).

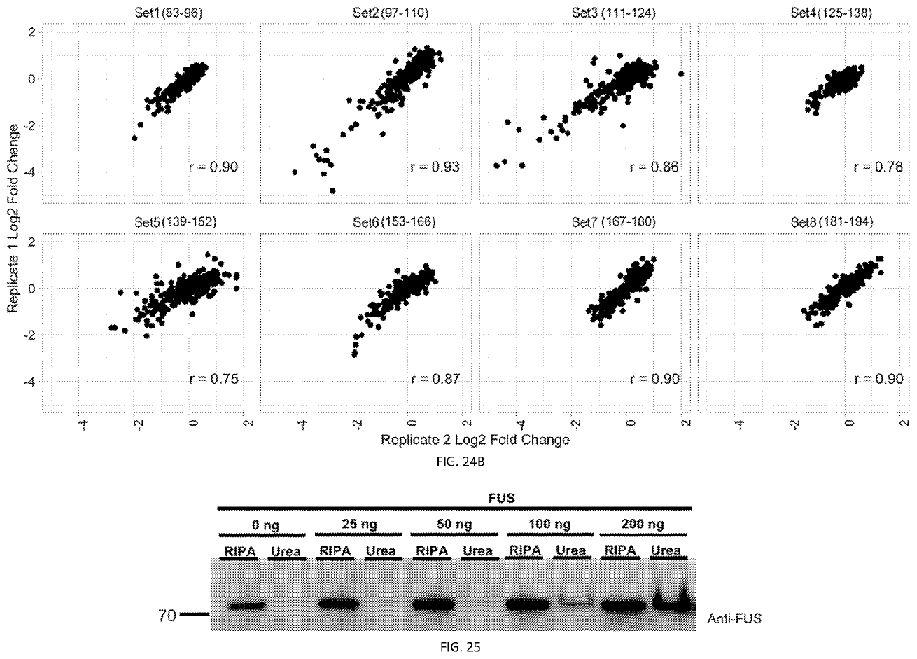

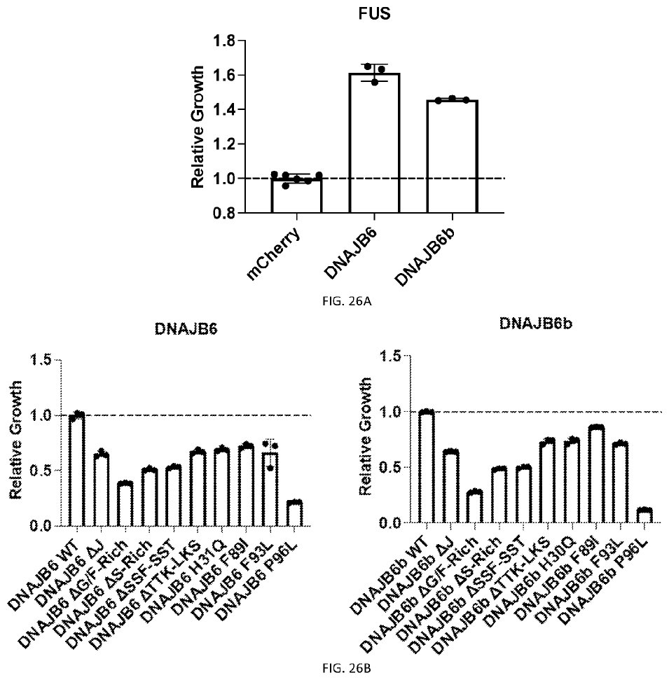

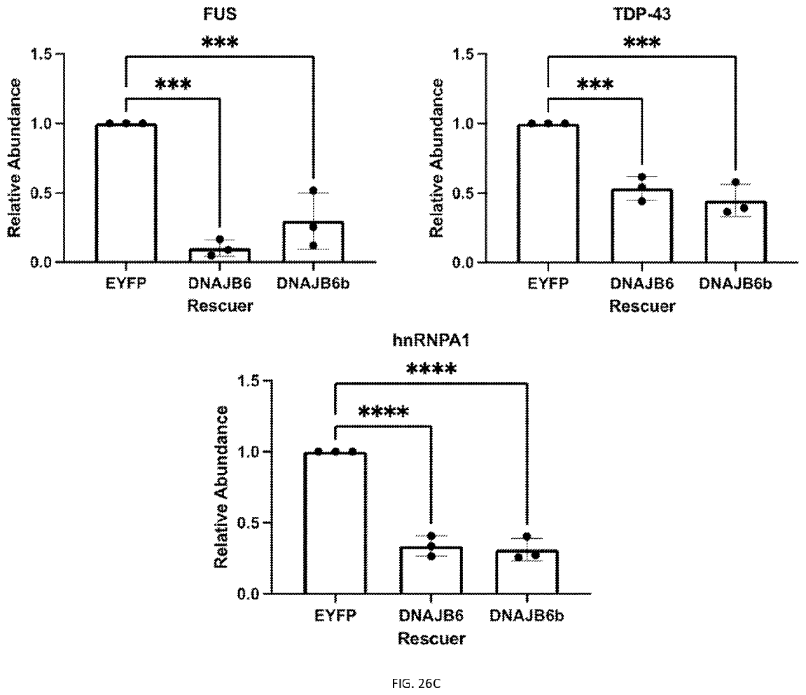

To further establish the significance of the enhanced DNAJB6 variants, their ability to reduce the formation of SDS-insoluble, urea-soluble FUS species was tested in mammalian HEK293T cells. Given that WT DNAJB6 almost entirely reduced the formation of SDS-insoluble, urea-soluble FUS species, transfecting more FUS expression plasmid resulted in higher levels of SDS-insoluble, urea-soluble FUS species (FIG. 25). Utilizing higher FUS expression, potentiated DNAJB6 variants, G182E and S192T, enabled DNAJB6 to further reduce the formation of SDS-insoluble, urea-soluble FUS species as compared to the wild-type protein (FIGS. 5D-5E).

### EXAMPLE 9

### Unbiased Screening of Human ORFs Identifies Known and Novel Rescuers of Proteotoxicity

To evaluate the performance of the platform against a larger, unbiased library of rescuers, the hORFeome V8.1 human cDNA library was screened against the pool of 35 models of neurodegeneration and protein misfolding. Human ORFs were chosen in hopes of identifying hits that are more likely to directly translate to mammalian models of disease and because of a paucity of prior reports of human genes being tested in this paradigm, suggesting that many novel interactions likely remain to be uncovered. Screening ˜900 members of the hORFeome library enabled examination of ˜40,000 interactions between the yeast models and human genes, representing ˜10 times more interactions surveyed within the system than is typical. Screening and subsequent validation of this collection of rescuers resulted in the identification of 54 confirmed genetic interactions. As expected with a library comprising random human genes, the occurrence of genetic interactions was significantly lower for this screen (0.135%) compared to the curated molecular chaperone screen (2.9%). Furthermore, when compared to other unbiased screens overexpressing yeast genes instead of human genes the hit rate remains lower (previously reported hit rates of 0.24%-1.13% vs. 0.135%). One of the main drivers of this difference is likely the failure of many human genes to function in yeast cells, and further highlights the benefits of the sensitive and quantitative screening approach which enables rapid exploration of this sparsely populated interaction space.

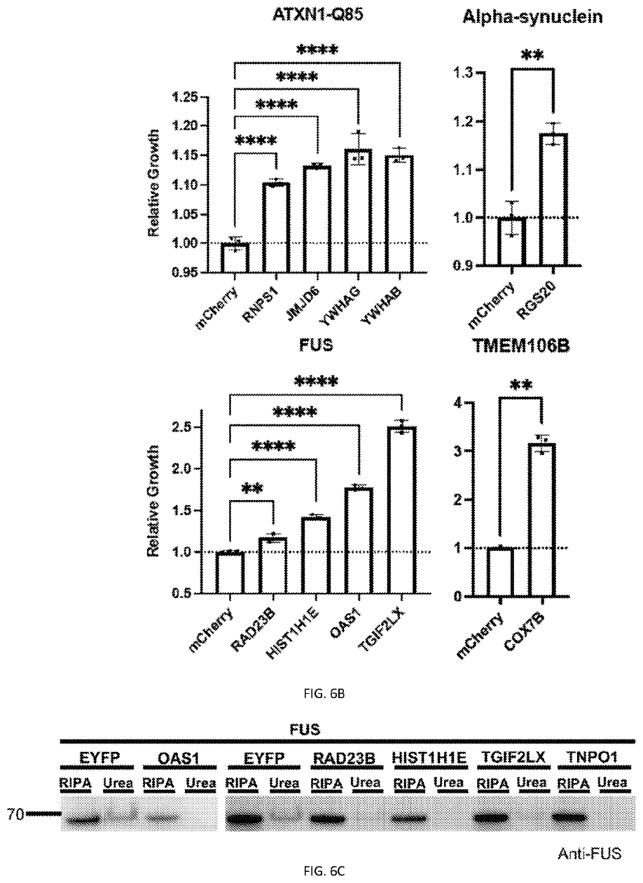

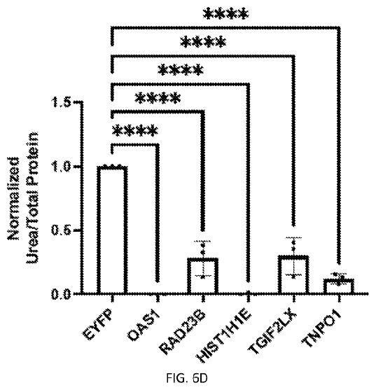

Despite a lower rate of interactions, rescuers were identified for a broad range of yeast models, including models that did not have rescuers from the initial molecular chaperone screen (FIG. 6A). A number of hits captured known interactions. For example, two 14-3-3 proteins, YWHAG and YWHAB were found to be rescuers of the ATXN1 polyglutamine (ATXNI-Q85) yeast model (FIG. 6B). Cytoplasmic 14-3-3 proteins bind ATXN1 via a motif centered around a phosphorylated serine at position 776, masking its NLS and diminishing the transport of ATXN1 into the nucleus which is necessary for ATXN1-Q85 toxicity. Other rescuers of ATXN1-Q85 in our screen were RNPS1 and JMJD6, two proteins involved in RNA-splicing, which ATXN1 also actively participates in (FIG. 6B). Furthermore, both RNPS1 and JMJD6 were recently identified as interactors of ATXN1-Q85 via pulldown and BIO-ID approaches, respectively. Two rescuers of the alpha-synuclein expressing model were also identified, one of which is a member of the Regulators of G protein signaling (RGS) family, RGS20 (FIG. 6B). RGS family proteins are structural proteins that accelerate the GTPase activity of α-subunits in G coupled-protein receptors, reducing the duration of downstream signaling. A number of RGS family proteins have been implicated in the development of Parkinson's Disease including a study of RGS6 in which expression of RGS6 in SNc Dopaminergic Neurons suppressed Parkinson's Disease phenotypes in aged mice.

Within the larger screen, a number of rescuers of the FUS yeast model were also discovered (FIG. 6B). These rescuers included RAD23B, a ubiquitin-binding proteasome-shuttle protein localized to the nucleus that is suspected to undergo phase separation, HIST1H1E, a member of the H1 linker histone family with predicted disordered regions, TGIF2LX, a DNA-binding homeobox protein with predicted disordered regions, and OAS1, a stress granule localized 2′5′-oligoadenylate synthetase protein. All of these rescuers demonstrated the ability to reduce the presence of SDS-insoluble FUS species upon co-overexpression with FUS in HEK293T cells (FIGS. 6C-6D). These results are particularly noteworthy as these rescuers performed similarly to a previously identified and extensively validated nuclear shuttling protein TNPO1 in their ability to reduce the presence of SDS-insoluble FUS (FIGS. 6C-6D).

### EXAMPLE 10

### FUS-Mediated Frontotemporal Dementia/Amyotrophic Lateral Sclerosis

To translate the DNAJB6 findings into in vivo contexts, a mouse model of FUS-mediated frontotemporal dementia/amyotrophic lateral sclerosis (FTD/ALS) is used. This animal model contains two copies of mutant FUS and shows a selective loss of motor neurons which is similar to that seen in patients. To deliver DNAJB6 into this animal model, a series of adeno-associated virus (AAV) vectors containing DNAJB6 under either a ubiquitous promoter such as CAGGS or Ubiquitin or a more narrow expression promoter such as the Synapsin 1 or Calcium/calmodulin-dependent protein kinase II promoter have been generated. As precise regulation of DNAJB6 levels may be important to obtain a therapeutic effect the vectors have also been designed with various 3′ UTR modifications to regulate RNA stability and translation rate. These viral vectors are injected into FTD/ALS models at post-natal day 0-2 at various doses and the effect on neuronal loss is assessed periodically via quantification of ChAT positive motor neurons in the lumbar spinal levels L4 and L5 in serial sections using confocal microscopy. Neuromuscular junction (NMJ) innervation is quantified by measuring pre-synaptic synaptophysin staining with fluorescent post-synaptic bungarotoxin. The activity of the enhanced DNAJB6 variants identified from the deep mutational scanning study is tested within in vivo contexts by moving them into AAV vectors and determining if they can provide additional benefit.

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

### REFERENCES

1 Hebert, L. E., Weuve, J., Scherr, P. A. & Evans, D. A. Alzheimer disease in the United States (2010-2050) estimated using the 2010 census. Neurology 80, 1778-1783, doi:10.1212/WNL.0b013e31828726f5 (2013).
2 Vanni, S., Colini Baldeschi, A., Zattoni, M. & Legname, G. Brain aging: A Ianus-faced player between health and neurodegeneration. J Neurosci Res 98, 299-311, doi:10.1002/jnr.24379 (2020).
3 Cavazzoni, P. The Path Forward: Advancing Treatments and Cures for Neurodegenerative Diseases, <fda.gov/news-events/congressional-testimony/path-forward-advancing treatments-and-cures-neurodegenerative-diseases-07292021> (2021).
4 Labbadia, J. & Morimoto, R. I. The biology of proteostasis in aging and disease. Annu Rev Biochem 84, 435-464, doi:10.1146/annurev-biochem-060614-033955 (2015).
5 Hipp, M. S., Kasturi, P. & Hartl, F. U. The proteostasis network and its decline in ageing. Nat Rev Mol Cell Biol 20, 421-435, doi:10.1038/s41580-019-0101-y (2019).
6 Outeiro, T. F. & Lindquist, S. Yeast cells provide insight into alpha-synuclein biology and pathobiology. Science 302, 1772-1775, doi:10.1126/science.1090439 (2003).
7 Armakola, M. et al. Inhibition of RNA lariat debranching enzyme suppresses TDP-43 toxicity in ALS disease models. Nat Genet 44, 1302-1309, doi:10.1038/ng.2434 (2012).
8 Sun, Z. et al. Molecular determinants and genetic modifiers of aggregation and toxicity for the ALS disease protein FUS/TLS. PLoS Biol 9, e1000614, doi:10.1371/journal.pbio.1000614 (2011).
9 Gitler, A. D. Beer and bread to brains and beyond: can yeast cells teach us about neurodegenerative disease? Neurosignals 16, 52-62, doi:10.1159/000109759 (2008).
10 Kim, H. J. et al. Therapeutic modulation of eIF2α phosphorylation rescues TDP-43 toxicity in amyotrophic lateral sclerosis disease models. Nat Genet 46, 152-160, doi:10.1038/ng.2853 (2014).
11 Cooper, A. A. et al. Alpha-synuclein blocks ER-Golgi traffic and Rabl rescues neuron loss in Parkinson's models. Science 313, 324-328, doi:10.1126/science.1129462 (2006).
12 Couthouis, J. et al. A yeast functional screen predicts new candidate ALS disease genes. Proc Natl Acad Sci U S A 108, 20881-20890, doi:10.1073/pnas.1109434108 (2011).
13 Kryndushkin, D., Ihrke, G., Piermartiri, T. C. & Shewmaker, F. A yeast model of optineurin proteinopathy reveals a unique aggregation pattern associated with cellular toxicity. Mol Microbiol 86, 1531-1547, doi:10.1111/mmi.12075 (2012).
14 Treusch, S. et al. Functional links between Aβ toxicity, endocytic trafficking, and Alzheimer's disease risk factors in yeast. Science 334, 1241-1245, doi:10.1126/science.1213210 (2011).
15 Ju, S. et al. A yeast model of FUS/TLS-dependent cytotoxicity. PLoS Biol 9, e1001052, doi:10.1371/journal.pbio.1001052 (2011).
16 Chen, Y. C. et al. Randomized CRISPR-Cas Transcriptional Perturbation Screening Reveals Protective Genes against Alpha-Synuclein Toxicity. Mol Cell 68, 247-257.e245, doi:10.1016/j.molcel.2017.09.014 (2017).
17 Lee, J. C. et al. Inhibition of p38 MAP kinase as a therapeutic strategy. Immunopharmacology 47, 185-201, doi:10.1016/s0162-3109(00)00206-x (2000).
18 Jo, M. et al. Yeast genetic interaction screen of human genes associated with amyotrophic lateral sclerosis: identification of MAP2K5 kinase as a potential drug target. Genome Res 27, 1487-1500, doi:10.1101/gr.211649.116 (2017).
19 Guerrero, E. N. et al. TDP-43/FUS in motor neuron disease: Complexity and challenges. Prog Neurobiol 145-146, 78-97, doi:10.1016/j.pneurobio.2016.09.004 (2016).
20 Yan, Z. et al. Yeast Barcoders: a chemogenomic application of a universal donor-strain collection carrying bar-code identifiers. Nat Methods 5, 719-725, doi: 10.1038/nmeth.1231 (2008).
21 Schmierer, B. et al. CRISPR/Cas9 screening using unique molecular identifiers. Mol Syst Biol 13, 945, doi:10.15252/msb.20177834 (2017).
22 Kim, H. J. et al. Mutations in prion-like domains in hnRNPA2B1 and hnRNPA1 cause multisystem proteinopathy and ALS. Nature 495, 467-473, doi:10.1038/nature11922 (2013).
23 Suzuki, H. & Matsuoka, M. The Lysosomal Trafficking Transmembrane Protein 106B Is Linked to Cell Death. J Biol Chem 291, 21448-21460, doi:10.1074/jbc.M116.737171 (2016).
24 Jovičić, A. et al. Modifiers of C9orf72 dipeptide repeat toxicity connect nucleocytoplasmic transport defects to FTD/ALS. Nat Neurosci 18, 1226-1229, doi:10.1038/nn.4085 (2015).
25 Zheng, C., Geetha, T. & Babu, J. R. Failure of ubiquitin proteasome system: risk for neurodegenerative diseases. Neurodegener Dis 14, 161-175, doi:10.1159/000367694 (2014).
26 Arndt, V., Rogon, C. & Hohfeld, J. To be, or not to be—molecular chaperones in protein degradation. Cell Mol Life Sci 64, 2525-2541, doi:10.1007/s00018-007-7188-6 (2007).
27 Jackrel, M. E. et al. Potentiated Hsp104 variants antagonize diverse proteotoxic misfolding events. Cell 156, 170-182, doi:10.1016/j.cell.2013.11.047 (2014).
28 Ioakeimidis, F. et al. A splicing mutation in the novel mitochondrial protein DNAJC11 causes motor neuron pathology associated with cristae disorganization, and lymphoid abnormalities in mice. PLoS One 9, e104237, doi:10.1371/journal.pone.0104237 (2014).
29 Chen, X. & Petranovic, D. Amyloid-β peptide-induced cytotoxicity and mitochondrial dysfunction in yeast. FEMS Yeast Res 15, doi:10.1093/femsyr/fov061 (2015).
30 Keefer, K. M., Stein, K. C. & True, H. L. Heterologous prion-forming proteins interact to cross-seed aggregation in Saccharomyces cerevisiae. Scientific Reports 7, 5853, doi:10.1038/s41598-017-05829-5 (2017).
31 Higurashi, T., Hines, J. K., Sahi, C., Aron, R. & Craig, E. A. Specificity of the J-protein Sis1 in the propagation of 3 yeast prions. Proceedings of the National Academy of Sciences 105, 16596-16601, doi: 10.1073/pnas.0808934105 (2008).
32 Breslow, D. K. et al. A comprehensive strategy enabling high-resolution functional analysis of the yeast genome. Nat Methods 5, 711-718, doi:10.1038/nmeth.1234 (2008).
33 Park, S. K. et al. Development and validation of a yeast high-throughput screen for inhibitors of Aβ₄₂ oligomerization. Dis Model Mech 4, 822-831, doi:10.1242/dmm.007963 (2011).
34 Sarnoski, E. A., Liu, P. & Acar, M. A High-Throughput Screen for Yeast Replicative Lifespan Identifies Lifespan-Extending Compounds. Cell Rep 21, 2639-2646, doi:10.1016/j.celrep.2017.11.002 (2017).
35 Wong, L. H. et al. A yeast chemical genetic screen identifies inhibitors of human telomerase. Chem Biol 20, 333-340, doi:10.1016/j.chembiol.2012.12.008 (2013).
36 Zhang, L. et al. A high-throughput screen for chemical inhibitors of exocytic transport in yeast. Chembiochem 11, 1291-1301, doi:10.1002/cbic.200900681 (2010).
37 Uhlen, M. et al. Proteomics. Tissue-based map of the human proteome. Science 347, 1260419, doi:10.1126/science.1260419 (2015).
38 Thiruvalluvan, A. et al. DNAJB6, a Key Factor in Neuronal Sensitivity to Amyloidogenesis. Mol Cell 78, 346-358.e349, doi:10.1016/j.molcel.2020.02.022 (2020).
39 Brehme, M. et al. A chaperome subnetwork safeguards proteostasis in aging and neurodegenerative disease. Cell Rep 9, 1135-1150, doi:10.1016/j.celrep.2014.09.042 (2014).
40 Fan, C. Y., Lee, S. & Cyr, D. M. Mechanisms for regulation of Hsp70 function by Hsp40. Cell Stress Chaperones 8, 309-316, doi:10.1379/1466-1268(2003)008<0309: mfrohf>2.0.co;2 (2003).
41 Kampinga, H. H. & Craig, E. A. The HSP70 chaperone machinery: J proteins as drivers of functional specificity. Nat Rev Mol Cell Biol 11, 579-592, doi:10.1038/nrm2941 (2010).
42 Gillis, J. et al. The DNAJB6 and DNAJB8 protein chaperones prevent intracellular aggregation of polyglutamine peptides. J Biol Chem 288, 17225-17237, doi:10.1074/jbc.M112.421685 (2013).
43 Kakkar, V. et al. The S/T-Rich Motif in the DNAJB6 Chaperone Delays Polyglutamine Aggregation and the Onset of Disease in a Mouse Model. Mol Cell 62, 272-283, doi:10.1016/j.molcel.2016.03.017 (2016).
44 Mansson, C. et al. DNAJB6 is a peptide-binding chaperone which can suppress amyloid fibrillation of polyglutamine peptides at substoichiometric molar ratios. Cell Stress Chaperones 19, 227-239, doi:10.1007/s12192-013-0448-5 (2014).
45 Hageman, J. et al. A DNAJB chaperone subfamily with HDAC-dependent activities suppresses toxic protein aggregation. Mol Cell 37, 355-369, doi: 10.1016/j.molcel.2010.01.001 (2010).
46 Harms, M. B. et al. Exome sequencing reveals DNAJB6 mutations in dominantly-inherited myopathy. Ann Neurol 71, 407-416, doi:10.1002/ana.22683 (2012).
47 Bengoechea, R., Pittman, S. K., Tuck, E. P., True, H. L. & Weihl, C. C. Myofibrillar 860 disruption and RNA-binding protein aggregation in a mouse model of limb-girdle muscular dystrophy 1D. Hum Mol Genet 24, 6588-6602, doi:10.1093/hmg/ddv363 (2015).
48 Chen, H. J. et al. The heat shock response plays an important role in TDP-43 clearance: evidence for dysfunction in amyotrophic lateral sclerosis. Brain 139, 1417-1432, doi:10.1093/brain/aww028 (2016).
49 Udan-Johns, M. et al. Prion-like nuclear aggregation of TDP-43 during heat shock is regulated by HSP40/70 chaperones. Hum Mol Genet 23, 157-170, doi:10.1093/hmg/ddt408 (2014).
50 Li, Y. R., King, O. D., Shorter, J. & Gitler, A. D. Stress granules as crucibles of ALS pathogenesis. J Cell Biol 201, 361-372, doi:10.1083/jcb.201302044 (2013).
51 Lin, Y., Protter, D. S., Rosen, M. K. & Parker, R. Formation and Maturation of Phase-Separated Liquid Droplets by RNA-Binding Proteins. Mol Cell 60, 208-219, doi:10.1016/j.molcel.2015.08.018 (2015).
52 Baradaran-Heravi, Y., Van Broeckhoven, C. & van der Zee, J. Stress granule mediated protein aggregation and underlying gene defects in the FTD-ALS spectrum. Neurobiol Dis 134, 104639, doi:10.1016/j.nbd.2019.104639 (2020).
53 Nizhnikov, A. A. et al. Proteomic screening for amyloid proteins. PLoS One 9, e116003, doi:10.1371/journal.pone.0116003 (2014).
54 Thibeault, J. et al. Sarkosyl: A milder detergent than SDS for identifying proteins with moderately high hyperstability using gel electrophoresis. Anal Biochem 571, 21-24, doi:10.1016/j.ab.2019.02.008 (2019).
55 Khong, A. et al. The Stress Granule Transcriptome Reveals Principles of mRNA Accumulation in Stress Granules. Mol Cell 68, 808-820.e805, doi:10.1016/j.molcel.2017.10.015 (2017).
56 Markmiller, S. et al. Context-Dependent and Disease-Specific Diversity in Protein Interactions within Stress Granules. Cell 172, 590-604.e513, doi:10.1016/j.cell.2017.12.032 (2018).
57 Patel, A. et al. A Liquid-to-Solid Phase Transition of the ALS Protein FUS Accelerated by Disease Mutation. Cell 162, 1066-1077, doi:10.1016/j.cell.2015.07.047 (2015).
58 Gu, J. et al. Hsp40 proteins phase separate to chaperone the assembly and maintenance of membraneless organelles. Proc Natl Acad Sci U S A 117, 31123-31133, doi:10.1073/pnas.2002437117 (2020).
59 Kachroo, A. H. et al. Evolution. Systematic humanization of yeast genes reveals conserved functions and genetic modularity. Science 348, 921-925, doi:10.1126/science.aaa0769 (2015).
60 Chen, H. K. et al. Interaction of Akt-phosphorylated ataxin-1 with 14-3-3 mediates neurodegeneration in spinocerebellar ataxia type 1. Cell 113, 457-468, doi:10.1016/s0092-8674(03)00349-0(2003).
61 Lai, S., O'Callaghan, B., Zoghbi, H. Y. & Orr, H. T. 14-3-3 Binding to ataxin-1(ATXN1) regulates its dephosphorylation at Ser-776 and transport to the nucleus. J Biol Chem 286, 34606-34616, doi:10.1074/jbc.M111.238527 (2011).
62 Zhang, S. et al. The ataxin-1 interactome reveals direct connection with multiple disrupted nuclear transport pathways. Nat Commun 11, 3343, doi:10.1038/s41467-020-17145-0 (2020).
63 Ahlers-Dannen, K. E., Spicer, M. M. & Fisher, R. A. RGS Proteins as Critical Regulators of Motor Function and Their Implications in Parkinson's Disease. Mol Pharmacol 98, 730-738, doi:10.1124/mol.119.118836 (2020)
64 Luo, Z. et al. Age-dependent nigral dopaminergic neurodegeneration and α-synuclein accumulation in RGS6-deficient mice. JCI Insight 5, doi:10.1172/jci.insight.126769 (2019).
65 Guo, L. et al. Nuclear-Import Receptors Reverse Aberrant Phase Transitions of RNA-Binding Proteins with Prion-like Domains. Cell 173, 677-692.e620, doi:10.1016/j.cell.2018.03.002 (2018).
66 Hofweber, M. et al. Phase Separation of FUS Is Suppressed by Its Nuclear Import Receptor and Arginine Methylation. Cell 173, 706-719.e713, doi:10.1016/j.cell.2018.03.004 (2018).
67 Qamar, S. et al. FUS Phase Separation Is Modulated by a Molecular Chaperone and Methylation of Arginine Cation-π Interactions. Cell 173, 720-734.e715, doi:10.1016/j.cell.2018.03.056 (2018).
68 Yoshizawa, T. et al. Nuclear Import Receptor Inhibits Phase Separation of FUS through Binding to Multiple Sites. Cell 173, 693-705.e622, doi:10.1016/j.cell.2018.03.003 (2018).
69 Weston, S. et al. A Yeast Suppressor Screen Used To Identify Mammalian SIRT1 as a Proviral Factor for Middle East Respiratory Syndrome Coronavirus Replication. J Virol 93, doi:10.1128/jvi.00197-19 (2019).
70 Fernandez-Acero, T. et al. A yeast-based in vivo bioassay to screen for class I phosphatidylinositol 3-kinase specific inhibitors. J Biomol Screen 17, 1018-1029, doi:10.1177/1087057112450051 (2012).
71 Murakami, T. et al. ALS/FTD Mutation-Induced Phase Transition of FUS Liquid Droplets and Reversible Hydrogels into Irreversible Hydrogels Impairs RNP Granule Function. Neuron 88, 678-690, doi:10.1016/j.neuron.2015.10.030 (2015).
72 Conicella, A. E., Zerze, G. H., Mittal, J. & Fawzi, N. L. ALS Mutations Disrupt Phase Separation Mediated by a-Helical Structure in the TDP-43 Low-Complexity C-Terminal Domain. Structure 24,1537-1549, doi:10.1016/j.str.2016.07.007 (2016).
73 Tosatto, L. et al. Single-molecule FRET studies on alpha-synuclein oligomerization of Parkinson's disease genetically related mutants. Sci Rep 5, 16696, doi:10.1038/srep16696 (2015).
74 Alberti, S. & Dormann, D. Liquid-Liquid Phase Separation in Disease. Annu Rev Genet 53, 171-194, doi:10.1146/annurev-genet-112618-043527 (2019).
75 Yu, H. et al. HSP70 chaperones RNA-free TDP-43 into anisotropic intranuclear liquid spherical shells. Science 371, doi:10.1126/science.abb4309 (2021).
76 Aprile, F. A. et al. The molecular chaperones DNAJB6 and Hsp70 cooperate to suppress α-synuclein aggregation. Sci Rep 7, 9039, doi:10.1038/s41598-017-08324-z (2017).
77 Mansson, C. et al. Interaction of the molecular chaperone DNAJB6 with growing amyloid-beta 42(Aβ42) aggregates leads to sub-stoichiometric inhibition of amyloid formation. J Biol Chem 289,31066-31076, doi:10.1074/jbc.M114.595124 (2014).
78 Li, W. et al. MAGeCK enables robust identification of essential genes from genome-scale CRISPR/Cas9 knockout screens. Genome Biol 15, 554, doi:10.1186/s13059-014-0554-4 (2014).
79 Brinkman, E. K., Chen, T., Amendola, M. & van Steensel, B. Easy quantitative assessment of genome editing by sequence trace decomposition. Nucleic Acids Res 42, e168,doi:10.1093/nar/gku936 (2014).
80 Sondheimer, N., Lopez, N., Craig, E. A. & Lindquist, S. The role of Sis1 in the maintenance of the [RNQ+] prion. Embo j 20, 2435-2442, doi:10.1093/emboj/20.10.2435 (2001).
81 Bagriantsev, S. N., Gracheva, E. O., Richmond, J. E. & Liebman, S. W. Variant-specific [PSI+] infection is transmitted by Sup35 polymers within [PSI+] aggregates with heterogeneous protein composition. Mol Biol Cell 19, 2433-2443, doi:10.1091/mbc.e08-01-0078 (2008).
82 Barlow, J. T., Bogatyrev, S. R. & Ismagilov, R. F. A quantitative sequencing framework for absolute abundance measurements of mucosal and lumenal microbial communities. Nat Commun 11, 2590, doi:10.1038/s41467-020-16224-6 (2020).
83 McIntyre, L. M. et al. RNA-seq: technical variability and sampling. BMC Genomics 12, 293, doi:10.1186/1471-2164-12-293 (2011).

1. A method for rescuing RNA-binding protein misfolding or preventing RNA-binding protein aggregation comprising contacting the RNA-binding protein with an effective amount of DNAJB6 or a variant thereof,
wherein the RNA-binding protein comprises Fused in Sarcoma (FUS), TAR DNA-binding protein (TDP-43), heterogeneous nuclear ribonucleoprotein A1 (hnRNPA1), or a combination thereof.

2. The method of claim 1, wherein the RNA-binding protein is in a cell and the contacting comprising providing an effective amount DNAJB6 or a variant thereof, or a nucleic acid encoding the DNAJB6 or variant thereof to the cell.

3. The method of claim 2, wherein the cell is in a subject and the contacting comprises administering an effective amount DNAJB6 or a variant thereof, or a nucleic acid encoding the DNAJB6 or a variant thereof to the subject.

4. The method of claim 2, further comprising decreasing the RNA-binding protein expression.

5. The method of claim 1, further comprising contacting the RNA-binding protein with an HSP70 family member protein.

6. A method for treating a disease or disorder characterized by protein misfolding or protein aggregation comprising administering an effective amount of DNAJB6 or a variant thereof, or a nucleic acid encoding the DNAJB6 or variant thereof to a subject in need thereof.

7. The method of claim 6, wherein the disease or disorder is characterized by protein misfolding or protein aggregation of an RNA-binding protein.

8. The method of claim 7, wherein the RNA-binding protein comprises Fused in Sarcoma (FUS), TAR DNA-binding protein (TDP-43), heterogeneous nuclear ribonucleoprotein A1 (hnRNPA1), or a combination thereof.

9. The method of claim 7, further comprising decreasing the RNA-binding protein expression.

10. The method of claim 6, wherein the disease or disorder is a neurodegenerative disease.

11. The method of claim 6, wherein the disease or disorder is selected from amyotrophic lateral sclerosis and frontotemporal dementia.

12. The method of claim 6, further comprising administering an HSP70 family member protein, or a nucleic acid encoding the HSP70 family member protein.

13. The method of claim 6, wherein the DNAJB6 comprises an amino acid sequence having at least 70% identity to SEQ ID NO:1 or SEQ ID NO: 12.

14. A DNAJB6 variant comprising an amino acid sequence having one or more amino acid substitutions at positions 101, 107, 140, 160, 171, 182, and 192 in reference to SEQ ID NO:1 or SEQ ID NO: 12.

15. (canceled)

16. The method of claim 1, wherein the DNAJB6 comprises an amino acid sequence having at least 70% identity to SEQ ID NO:1 or SEQ ID NO: 12.

17. The method of claim 13, wherein the DNAJB6 comprises an amino acid sequence having one or more amino acid substitutions at positions 101, 107, 140, 160, 171, 182, and 192 in reference to SEQ ID NO:1 or SEQ ID NO: 12.

18. The method of claim 17, wherein the DNAJB6 comprises one or more mutations of R101, R107, F140, G160, G171, G182, and S192, as compared to SEQ ID NO:1 or SEQ ID NO: 12.

19. The method of claim 16, wherein the DNAJB6 comprises an amino acid sequence having one or more amino acid substitutions at positions 101, 107, 140, 160, 171, 182, and 192 in reference to SEQ ID NO:1 or SEQ ID NO: 12.

20. The method of claim 19, wherein the DNAJB6 comprises one or more mutations of R101, R107, F140, G160, G171, G182, and S192, as compared to SEQ ID NO:1 or SEQ ID NO: 12.

21. The method of claim 2, wherein the cell is a neuronal cell or glial cell.


SEQUENCE LISTING (12 sequences)

SEQ ID NO: 1 - protein, 326 aa, Homo sapiens
MVDYYEVLGVQRHASPEDIKKAYRKLALKWHPDKNPENKEEAERKFKQVAEAYEVLSDAKKRDIYDKYGKEGLNGGGGGGSHFDSPFEFGFTFRNPDDVFREFFGGRDPFSFDFFEDPFEDFFGNRRGPRGSRSRGTGSFFSAFSGFPSFGSGFSSFDTGFTSFGSLGHGGLTSFSSTSFGGSGMGNFKSISTSTKMVNGRKITTKRIVENGQERVEVEEDGQLKSLTINGVADDDALAEERMRRGQNALPAQPAGLRPPKPPRPASLLRHAPHCLSEEEGEQDRPRAPGPWDPLASAAGLKEGGKRKKQKQREESKKKKSTKGNH

SEQ ID NO: 2 - DNA, 20 bp, synthetic construct
  source 1..20: mol_type = other DNA
gcatatgaagtgctgtcgga

SEQ ID NO: 3 - DNA, 20 bp, synthetic construct
  source 1..20: mol_type = other DNA
gacttctttgggaatcgaag

SEQ ID NO: 4 - DNA, 20 bp, synthetic construct
  source 1..20: mol_type = other DNA
aaaaagcttccgcctgatgg

SEQ ID NO: 5 - DNA, 20 bp, synthetic construct
  source 1..20: mol_type = other DNA
aaaacaggacgatgtgcggc

SEQ ID NO: 6 - protein, 21 aa, synthetic construct
FDSPFEEFGFTFRNPDDVFRE

SEQ ID NO: 7 - protein, 21 aa, synthetic construct
XDSPFEEFGFTFRNPDDVFRE

SEQ ID NO: 8 - protein, 21 aa, synthetic construct
FXSPFEEFGFTFRNPDDVFRE

SEQ ID NO: 9 - protein, 21 aa, synthetic construct
FDXPFEEFGFTFRNPDDVFRE

SEQ ID NO: 10 - protein, 21 aa, synthetic construct
FDSXFEEFGFTFRNPDDVFRE

SEQ ID NO: 11 - protein, 21 aa, synthetic construct
FDSPXEEFGFTFRNPDDVFRE

SEQ ID NO: 12 - protein, 241 aa, synthetic construct
MVDYYEVLGVQRHASPEDIKKAYRKLALKWHPDKNPENKEEAERKFKQVAEAYEVLSDAKKRDIYDKYGKEGLNGGGGGGSHFDSPFEFGFTFRNPDDVFREFFGGRDPFSFDFFEDPFEDFFGNRRGPRGSRSRGTGSFFSAFSGFPSFGSGFSSFDTGFTSFGSLGHGGLTSFSSTSFGGSGMGNFKSISTSTKMVNGRKITTKRIVENGQERVEVEEDGQLKSLTINGKEQLLRLDNK